(19) United States
(12) Patent Application Publication
(10) Pub. No.: US 2020/0308538 A1
(43) Pub. Date: Oct. 1, 2020

(54) COMPOSITIONS AND METHODS FOR TREATING LIVER DISEASE AND DYSFUNCTION

(71) Applicants: BETH ISRAEL DEACONESS MEDICAL CENTER, INC., BOSTON, MA (US); VIRGINIA COMMONWEALTH UNIVERSITY, RICHMOND, VA (US)

(72) Inventors: ROBERT A. FISHER, BOSTON, MA (US); GIUSEPPE PETTINATO, BOSTON, MA (US)

(73) Assignees: BETH ISRAEL DEACONESS MEDICAL CENTER, INC., BOSTON, MA (US); VIRGINIA COMMONWEALTH UNIVERSITY, RICHMOND, VA (US)

(21) Appl. No.: 16/644,903

(22) PCT Filed: Sep. 10, 2018
(86) PCT No.: PCT/US2018/050254

§ 371 Date: Mar. 5, 2020

Related U.S. Application Data
(60) Provisional application No. 62/557,533, filed on Sep. 12, 2017.

(51) Int. Cl. C12N 5/071; A61K 35/407

(52) U.S. Cl. CPC C12N 5/067; A61K 35/407; C12N 2501/119; C12N 2501/155; C12N 2513/00; C12N 2501/12; C12N 2501/20; C12N 2506/45; C12N 2501/415

(57) ABSTRACT

The invention features compositions and methods that are useful for generating human hepatocyte-like cells (HLCs) and methods of using such cells for the treatment of diseases associated with a loss in liver cell number or function.

### CROSS-REFERENCE TO RELATED APPLICATION

This application claims the benefit of the following U.S. Provisional Application No. 62/557,533, filed Sep. 12, 2017, the entire contents of which are incorporated herein by reference.

### BACKGROUND OF THE INVENTION

Treatment of acute liver failure by cell transplantation is hindered by a shortage of human hepatocytes. Current protocols for hepatic differentiation of human induced pluripotent stem cells (hiPSCs) result in low yields, cellular heterogeneity, and limited scalability. Liver dysfunction that is caused by cirrhosis, hepatitis, or acute liver failure is frequently fatal. To date, the most effective therapy for acute liver failure is liver transplantation. However, donor liver shortages and the requirement for lifelong immunosuppression have limited the use of liver transplantation. As a result, hepatocyte transplantation and bioartificial liver (BAL) devices containing active hepatocytes that remove toxins and supply key physiological active molecules to sustain hepatic function have been used to bridge patients to native regeneration or organ transplantation. These therapeutic modalities, however, are limited by the lack of human livers as a source of hepatocytes and limitations of xenogenic sources. Additionally, practical limitations of hepatocyte-based therapies include the rapid deterioration in function of primary hepatocytes in culture, and their variable viability upon recovery from cryopreservation.

Human induced pluripotent stem cells hold great promise in personalized regenerative medicine due to their pluripotent potential, high proliferative index, and absence of rejection and ethical controversy. Induced pluripotent stem cells (iPSCs) can be generated by retro-engineering adult differentiated cells back into a pluripotent state through the addition of various stemness factors. hiPSCs demonstrate three-germ layer differentiation potential and can be differentiated into a wide variety of cell types, including hepatocyte-like cells (HLCs). HLCs that are derived from hiPSCs represent a promising, potentially inexhaustible alternative source of hepatocytes in cell therapy and bioengineered livers for the treatment of hepatic diseases, pharmaceutical testing, as well as the study of the developmental biology of hepatogenesis. Theoretically, hiPSC-derived hepatocytes have the potential to enable autologous cell transplantation and thereby mitigate the adverse effects of immune sensitization and rejection. The translational potential of stem cell-derived HLCs has not been realized due to the large cell doses required per transplantation. Current differentiation protocols for generating HLCs from hiPSCs are limited by low yields and cellular heterogeneity. Thus, there is a need for new compositions and methods related to the generation of HLCs from hiPSCs for regenerative medicine.

### SUMMARY OF THE INVENTION

The invention features compositions and methods that are useful for generating human hepatocyte-like cells (HLCs) and methods of using such cells for the treatment of diseases associated with a loss in liver cell number or function.

In a first aspect, the invention features a method for generating hepatocyte-like cells the method involving (a) incubating induced pluripotent stem cell in a round bottomed convex well comprising agarose to generate a spherical embryoid body, (b) contacting the embryoid body with one or more differentiation factors selected from the group consisting of basic FGF, Activin-A and TGF-β, thereby forming an embryoid body comprising definitive endoderm cells, (c) contacting the embryoid body of step b with FGF4 and/or BMP-4 to form an embryoid body comprising foregut endoderm cells, (d) contacting the embryoid body of step c with a Wnt pathway inhibitor to form an embryoid body comprising hepatoblast cells, and (e) contacting the embryoid body of step d with a HGF and/or Oncostatin A to form an embryoid body comprising mature hepatocyte-like cells.

In a second embodiment, the invention provides a method for generating hepatocyte-like cells the method involving (a) incubating an induced pluripotent stem cell and an adipose-tissue derived endothelial cell in a round bottomed convex well comprising agarose to generate a spherical embryoid body, (b) contacting the embryoid body with one or more differentiation factors selected from the group consisting of basic FGF, Activin-A and TGF-β, thereby forming an embryoid body comprising definitive endoderm cells, (c) contacting the embryoid body of step b with FGF4 and/or BMP-4 to form an embryoid body comprising foregut endoderm cells, (d) contacting the embryoid body of step c with a Wnt pathway inhibitor to form an embryoid body comprising hepatoblast cells, and (e) contacting the embryoid body of step d with a HGF and/or Oncostatin A to form an embryoid body comprising mature hepatocyte-like cells.

In various embodiments of any aspect delineated herein, the definitive endoderm cells express SOX17 and FOXA2, the foregut endoderm cells express HHEX and GATA4, the hepatoblast cells express AFP and HNF-4α, and the hepatocyte-like cells express one or more of the following markers ALBUMIN, HNF-1α, C-MET, and CK-18. In various embodiments of any aspect delineated herein, step b comprises contacting the embryoid body with basic FGF, Activin-A and TGF-β, step c comprises contacting the embryoid body of step b with FGF4 and BMP-4, or step c comprises contacting the embryoid body of step c with WIF-1 and DKK-1.

In various embodiments of any aspect delineated herein, the hepatocyte-like cells express five P450 isoforms Cyp1B1, Cyp2C9, Cyp3A4, Cyp2B6 and Cyp3A7. In some embodiments of any aspect delineated herein, the hepatocyte-like cells express Alpha fetoprotein, Albumin, and CK18. In other embodiments of any aspect delineated herein, the hepatocyte-like cells of step d form a cluster that is 800-1,000 μm, but that shows no core necrosis. In various embodiments of any aspect delineated herein, the hepatocyte-like cells display one or more of the following functional activities: acetylated low-density lipoprotein (DiI-ac-LDL) uptake, indocyanine green (ICG—Cardiogreen) absorption and release after 6 hours, glycogen storage, and cytoplasmic accumulation of neutral triglycerides and lipids.

In various embodiments of any aspect delineated herein, the hepatocyte-like cells are capable of ammonium metabolism. In various embodiments of any aspect delineated herein, detoxification as measured by increase in CYP isoform gene expression. In some embodiments, the hepatocyte-like cells secrete Albumin, Alpha Fetoprotein and/or fibrinogen. In various embodiments of any aspect delineated herein, the hepatocyte-like cells comprise intracellular Urea. In various embodiments of any aspect delineated herein, the method generates 80% or more hepatocyte-like cells. In some embodiments, the induced pluripotent stem cell and adipose-tissue derived endothelial cell are mammalian cells. In some embodiments, the induced pluripotent stem cell and adipose-tissue derived endothelial cell are rodent or human cells. In some embodiments, the induced pluripotent stem cell is derived from an amniotic cell.

In various embodiments of any aspect delineated herein, the hepatocyte-like cells form a cluster. In some embodiments, the method further comprises coating the cluster with a hydrogel and culturing the coated cluster with mesenchymal stem cells, thereby forming a mesenchymal layer of cells around the cluster. In some embodiments, the induced pluripotent stem cell and adipose-tissue derived endothelial cell are autologous or heterologous cells.

In various embodiments of any aspect delineated herein, the hepatocyte-like cells is capable of functioning in the Liver phase 1 and/or Liver phase 2 detoxification pathway. In some embodiments, the hepatocyte-like cell is capable secreting glutathione.

In various embodiments of any aspect delineated herein, the hepatocyte-like cell secretes a coagulation factor. In some embodiments, the coagulation factor is von Willebrand factor (vWF), Factor IX, Protein C, Factor X, Protein S, Factor V, Factor VIII, Antithrombin, Factor VII, Factor XI, C-reactive Protein, Factor XII, Prothrombin and Factor XIII.

In various embodiments of any aspect delineated herein, the invention provides a method for treating a blood coagulation disorder, the method comprising administering to a subject having the blood coagulation disorder a hepatocyte-like cell produced according to the method of any one of the aspects delineated herein. In some embodiments, the hepatocyte-like cell secretes a coagulation factor. In some embodiments, the coagulation factor is von Willebrand factor (vWF), Factor IX, Protein C, Factor X, Protein S, Factor V, Factor VIII, Antithrombin, Factor VII, Factor XI, C-reactive Protein, Factor XII, Prothrombin and Factor XIII. In some embodiments, the blood coagulation disorder is hemophilia.

In various embodiments of any aspect delineated herein, the invention provides a method for treating liver disease or dysfunction, the method comprising administering to a subject having liver disease or dysfunction a hepatocyte-like cell produced according to the method of any one of the aspects delineated herein. In some embodiments, the subject has acute liver failure, cirrhosis, hepatitis B or C infection, hepatocellular carcinoma, Crigler-Najjar Syndrome, Urea Cycle Defects, Ornithine Transcarbamylase (OTC) Deficiency, Carbamoyl-Phosphate Synthetase I (CPS-1) Deficiency, Citrullinemia (Cit) disorder, Arginosuccinate Lyase (ASL) Deficiency, Familial Hypercholesterolemia, Hemophilia, Factor VII, Glycogen storage disease, Phenylketonuria (PKU), Infantile Refsum Disease, Progressive Familial Intrahepatic Cholestasis (PFIC-2), AlAT Deficiency, or Primary Oxalosis. In some embodiments, the subject has end-stage liver disease.

In various embodiments of any aspect delineated herein, the invention provides a cellular composition comprising a hepatocyte-like cell produced according to the method of the first aspect delineated herein and an excipient. In various embodiments of any aspect delineated herein, the invention provides a cellular composition comprising a hepatocyte-like cell produced according to the method of the second aspect delineated herein and an excipient. In various embodiments of any aspect delineated herein, the induced pluripotent stem cell is derived from an amniotic cell. In various embodiments of any aspect delineated herein, the the hepatocyte-like cell secretes a coagulation factor. In some embodiments, the secretion is constitutive or inducible. In some embodiments, the coagulation factor is von Willebrand factor (vWF), Factor IX, Protein C, Factor X, Protein S, Factor V, Factor VIII, Antithrombin, Factor VII, Factor XI, C-reactive Protein, Factor XII, Prothrombin and Factor XIII.

In various embodiments of any aspect delineated herein, the invention provides a kit comprising a hepatocyte-like cell produced according to the method of claim 1 and instructions for the administration of said cell.

The invention provides cellular compositions comprising human hepatocyte-like cells (HLCs) and methods of using such cells for the treatment of disease. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. Agents conventionally administered to transplant recipients may optionally be used in connection with the cellular compositions described herein.

By “ameliorate” is meant decrease, suppress, attenuate, diminish, arrest, or stabilize the development or progression of a liver disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ, such as the liver. Examples of liver diseases include cirrhosis, hepatitis B or C infection, hepatocellular carcinoma, Crigler-Najjar Syndrome, Urea Cycle Defects, Ornithine Transcarbamylase (OTC) Deficiency, Carbamoyl-Phosphate Synthetase I (CPS-1) Deficiency, Citrullinemia (Cit) disorder, Arginosuccinate Lyase (ASL) Deficiency, Familial Hypercholesterolemia, Hemophilia, Factor VII, Glycogen storage disease, Phenylketonuria (PKU), Infantile Refsum Disease, Progressive Familial Intrahepatic Cholestasis (PFIC-2), A1AT Deficiency, and Primary Oxalosis.

By “effective amount” is meant the amount of a composition of the invention required to ameliorate the symptoms of a disease relative to an untreated patient. The effective amount of cells used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. Appropriate dosages of human hepatocytes administered by portal vein infusion is as follows: 30-100×10⁶ hepatocytes per kilogram of patient body weight, at an infusion rate of 5-10 ml/kg/hr, and a concentration of 1-10×10⁶ hepatocytes/1 ml, nonsteatotic hepatocytes suspended in Dextrose 5% in Lactated Ringers Solution (D5LR). Infusion takes place over 30-minute intervals, on ice, to maintain a mild hypothermic 32° C. solution temperature (Fisher R. A. et al., Cell Transplant 2004; 13(6): 677-689). Appropriate dosages of human hepatocytes administered by spleen injection and splenic artery infusion is as follows: no greater than 6×10⁸ hepatocytes per infusion, at an infusion speed, concentration and temperature as in portal infusion described above (Fisher R. A. et al., Hepatocyte Review, M N Berry and A M Edwards (eds.) 2000:475-501).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker” is meant any protein or polynucleotide having an alteration in expression level or activity that is associated with liver disease or the differentiation state of a liver cell, tissue or organ. Exemplary markers of hepatocyte differentiation are disclosed herein.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C show the differentiation of human induced pluripotent stem cell (hiPSC) embryoid bodies (hiPSC-EBs) in 3D culture into hepatocyte-like cells. FIG. 1A depicts a schematic representation of the 4-stage differentiation protocol and the major regulatory factors administered at each stage. The differentiation protocol recapitulates the stages of ontogenic development of liver. Starting from the undifferentiated human induced pluripotent stem cells (hiPSCs), the cells undergo differentiation to Definitive Endoderm (DE), followed by Foregut Endoderm (FE) from where the Hepatic Progenitor Cells (HPCs) or Hepatoblasts arise. The final maturation step leads to mature Hepatocyte-Like Cells (HLCs). FIG. 1B is a graph showing the hiPSC-EBs differentiated with both WIF-1 and DKK-1 and exhibited greater expressions of hepatocyte-specific markers relative to the ones differentiated without the WIF-1 and DKK-1. Data presented as mean±SD (n=3). FIG. 1C is a graph showing hiPSC-EBs differentiated without WIF-1 and DKK-1, and showed greater expressions of cholangiocyte-specific markers relative to the ones differentiated with both WIF-1 and DKK-1. Data presented as mean±SD (n=3).

FIGS. 2A-2F depict the stage-specific gene expressions and protein expressions of hiPSC-EBs during the differentiation process. FIG. 2A shows representative immunofluorescence images of hiPSC-EBs during the differentiation process. SOX17 and FOXA2 are markers for the definitive endoderm stage; HHEX and GATA4 are markers for the foregut endoderm; AFP and HNF-4α are markers for the hepatic progenitor cells; ALBUMIN and CK-18 are markers for the mature HLCs. DAPI stains for cell nuclei. Scale bar 100 μm. FIG. 2B is a graph depicting stage-specific gene expression analysis by Real-Time PCR. The relative quantities of stage-specific genes were measured at the mRNA level to follow the progression of the differentiation process. Sox17 as the definitive endoderm marker; Gata4 as the foregut endoderm marker; HNF-4α as the hepatic progenitor cells marker; Albumin was used to determine the final maturation for the hepatocyte-like cells (HLCs). Undifferentiated cells were used as negative control. FIG. 2C is a graph that depicts quantitative RT-PCR displaying the presence of mRNA for AFP, five P450 isoforms (Cyp3A4, Cyp2C9, Cyp3A7, Cyp1B1, and Cyp2B6), Albumin, and CK18 in the terminally differentiated hiPSC-EB-HLCs with and without inhibitors. Gene expression for the condition with inhibitors was greater compared with the one without inhibitors for any gene tested; FIG. 2D and FIG. 2E are representative immunofluorescence images that follow the differentiation program. Terminally differentiated hiPSC-EB-HLCs expressed mature hepatocyte-specific markers, as evidenced by co-staining of ALBUMIN and HNF-1α, and ALBUMIN and C-MET. Scale bar 100 μm. FIG. 2F is a FACS analysis for albumin positive cells showing a higher percentage of albumin producing cells in the condition with inhibitors compared with the one without inhibitors (80% vs 68%).

FIGS. 3A-3D depict four graphs showing the secretion of hepatic proteins by hiPSC-EB-HLCs. The conditioned medium from hiPSC-EB-HLCs was collected 48 hours following the completion of the differentiation process for both conditions with and without inhibitors. (FIG. 3A) Albumin, (FIG. 3B) Alpha Fetoprotein (AFP) and (FIG. 3C) fibrinogen were detected in the medium and (FIG. 3D) intracellular Urea was detected. The difference in secretion between the conditions with inhibitors was statistically significant with respect to the condition without inhibitors. Undifferentiated hiPSCs were used as negative control. The results are representative of at least three independent experiments. Data presented as mean±SD (n=3). *p<0.05; **p<0.01; ***p<0.001.

FIGS. 4A-4E depict representative images showing that the resultant hiPSC-EB-HLCs displayed functional activities typical of mature primary hepatocytes, such as (FIG. 4A) Acetylated low-density lipoprotein (DiI-ac-LDL) uptake; (FIG. 4B) Indocyanine green (ICG—Cardiogreen) uptake; (FIG. 4C) ICG release after 6 hours; (FIG. 4D) glycogen storage indicated by PAS staining; and (FIG. 4E) cytoplasmic accumulation of neutral triglycerides and lipids indicated by Oil-Red O staining for both conditions with and without inhibitors. Undifferentiated hiPSCs were used as negative control. Scale bar 100 μm.

FIGS. 5A-5D depict graphs showing CYP enzyme induction analysis comparing two experimental conditions with and without inhibitors. (FIG. 5A) Ammonium metabolism assay over a 24-hour period for both conditions with and without inhibitors; Cytochrome P450 (CYP450) induction analysis comparing the two experimental conditions with and without inhibitors. Several CYP enzymes were evaluated through incubation of the cells with different inducers: (FIG. 5B) Phenobarbital for the CYP2B6, (FIG. 5C) Rifampicin for the CYP3A4 and (FIG. 5D) Omeprazole for the CYP1A2 for a period of 72 hours. DMSO was used as control to test the basal activity of different CYP450. Data presented as mean±SD (n=3). *p<0.05; ***p<0.001.

FIGS. 6A-6F depict in vivo functionality of the hiPSC-EB-HLCs in a rat model of acute liver failure induced by D-Galactosamine. (FIG. 6A) Serum level of alanine aminotransferase (ALT). The mean values of ALT prior to liver injury was 53 U/L; after injury was significantly higher at 3781 U/L; and at 2 weeks was 78 U/L following transplantation of hiPSC-EB-HLCs treated with the two inhibitors, and 364 U/L for the hiPSC-EB-HLCs without inhibitors; (FIG. 6B) The Kaplan-Meier survivals were determined 14 days after cell transplantation; (FIG. 6C) Histological examination of the liver sections of the survived animals at 14 days after hiPSC-EB-HLCs transplantation showed intense positive staining for human albumin; 20× and 40×. FIG. 6D shows representative patterns of positive staining of human albumin in the livers of the hiPSC-EB-HLC transplantation group at 14 days post-transplantation. Spleen sections in all animals in this group were negative for human albumin staining. FIG. 6E is an immunofluorescence of the rat liver after transplantation and shows the co-staining of several human hepatic proteins such as HNF-3β, Albumin and C-MET. FIG. 6F shows immunofluorescence of human liver used as a positive control displaying staining of all three human hepatic proteins.

FIG. 7 depicts images showing that embryoid bodies were produced using an agarose micro-well arrays and Teflon stamps and without the need for rho-associated kinase inhibitors (ROCKi), and/or centrifugation (Rocki/Spin-free). An 80% confluent six-well plate containing 1.2×10⁶ dissociated hiPSC produced approximately 280 embryoid bodies. Scale bar 600 μm.

FIG. 8 depicts representative microscopic fields showing human albumin-producing cells (staining) after differentiation. Scale bar 200 μm.

FIG. 9 depicts representative microscopic fields showing that the hiPSC-EB-HLCs increased in size from approximately 500 μm after 24 hours of their formation to 800-1,000 μm at the end of differentiation process without any core necrosis at any time. The image shows a live-dead stain of a representative hiPSC-EB-HLC at the end of the differentiation process. Scale bar 200 μm.

FIG. 10 depicts two light microscopy images showing that hiPSC-EB-HLCs were morphologically polygonal with enriched cytoplasmic granules (arrows). The differentiated clusters were allowed to attach to a coated plate for morphological examination. Upper picture at 1 week after attachment, lower picture at 2 weeks after attachment. Scale bar 100 μm.

FIGS. 11A-11C depict three images of clusters after spreading onto a matrigel-coated plate, which showed a homogeneous distribution of the signal for each functional activity. FIG. 11A) Indocyanin green; FIG. 11B) glycogen storage; FIG. 11C) cytoplasmic accumulation of neutral triglycerides and lipids. Scale bar 200 μm.

FIGS. 12A-12D depict the immunofluorescence for various markers that were used to track and confirm differentiation into mature hepatocyte-like cells. SOX17 and FOXA2 are markers for the endodermal stage; HHEX and GATA4 for the foregut endoderm; AFP and HNF-4a for the hepatic progenitor cells; ALBUMIN and CK-18 for mature hepatocyte-like cells. FIG. 12A and FIG. 12B: Comparison for the maturation steps between human embryoid bodies (hEBs) with hiPSCs only (FIG. 12A) and hEBs with hiPSCs interlaced with human endothelial cells (hECs) (FIG. 12B) displaying the presence of stage specific markers. FIG. 12C is a FACS analysis for albumin between the two experimental conditions with and without hECs. FIG. 12D provides results of quantitative RT-PCR showing the effect of endothelial cells on gene expression.

FIGS. 13A-13D are bar graphs showing that albumin (FIG. 13A), fibrinogen (FIG. 13B), and alpha fetoprotein (AFP) (FIG. 13C) were secreted into the media in the presence and absence of endothelial cells. FIG. 13D shows the intracellular concentration of urea detected in the presence and absence of endothelial cells after differentiation.

FIGS. 14A-14E are images showing ICG uptake (FIG. 14A), ICG release (FIG. 14B), Oil-Red O Staining (FIG. 14C), PAS staining (FIG. 14D), and Dil-Ac-LDL Uptake (FIG. 14E), under different conditions (hiPSC-EB-HLC plus hEC, left column; hiPSC-EB-HLC no hEC, middle column; undifferentiated hiPSC, right column). Scale bar 100 μm.

FIGS. 15A-15D are graphs showing results of an ammonium metabolism assay (FIG. 15A), or CYP enzyme induction analysis comparing different experimental conditions (FIG. 15B, FIG. 15C, FIG. 15D). FIG. 15A shows results of an ammonium metabolism assay. The ammonium concentration was measured in the cell culture supernatant over a 24-hour period for both conditions (hiPSC-EB-HLC no hEC, hiPSC-EB-HLC plus hEC). FIGS. 15B-15D show several cytochromes P450 enzymes were evaluated by incubating the cells with different inducers: Omeprazole for CYP1A2 (FIG. 15B), Rifampicin for CYP3A4 (FIG. 15C), and Phenobarbital for CYP2B6 (FIG. 15D), over a 72-hour period. DMSO was used as control to test the basal activity of the different CYP450.

FIGS. 16A-16I show the therapeutic effects of hiPSC-EB-HLC in acute liver failure in an animal model. FIG. 16A shows a Kaplan-Meier survival curve for model assessment without transplantation. FIG. 16B shows a Kaplan-Meier survival plot of animals after hiPSC-EB-HLC with and without hECs transplantation. FIG. 16C, FIG. 16D, FIG. 16E, and FIG. 16F depict images of representative liver and spleen sections from sacrificed animals post-transplantation with hiPSC-EB-HLC with hECs using immunohistochemical staining. Background staining with hematoxylin. Scale bar 2.5 μm. FIG. 16G, FIG. 16H, and FIG. 16I show representative liver and spleen sections with immunofluorescence staining. Nuclear staining with DAPI. Scale bar 100 μm.

FIGS. 17A-17G are graphs showing coagulation factors analyzed in HLCs. iPSCs were compared to iPSC+EC. The coagulation factors analyzed include von Willebrand factor (vWF) and Factor IX (FIG. 17A), Protein C and Factor X (FIG. 17B), Protein S and Factor V (FIG. 17C), Factor VIII and Antithrombin (FIG. 17D), Factor VII and Factor XI (FIG. 17E), C-reactive Protein and Factor XII (FIG. 17F), and Prothrombin and Factor XIII (FIG. 17G).

### DETAILED DESCRIPTION OF THE INVENTION

The invention features compositions and methods that are useful for generating human hepatocyte-like cells (HLCs) and methods of using such cells for the treatment of diseases associated with a loss in liver cell number or function.

The invention is based, at least in part, on the discovery of a novel multicellular spheroid-based hepatic differentiation protocol starting from embryoid bodies of hiPSCs (hiPSC-EBs) for robust mass production of human hepatocyte-like cells (HLCs) using two novel inhibitors of the Wnt pathway. The resultant hiPSC-EB-HLCs expressed liver-specific genes, secreted hepatic proteins such as Albumin, Alpha Fetoprotein, and Fibrinogen, metabolized ammonia, and displayed cytochrome P450 activities and functional activities typical of mature primary hepatocytes, such as LDL storage and uptake, ICG uptake and release, and glycogen storage. Cell transplantation of hiPSC-EB-HLC in a rat model of acute liver failure significantly prolonged the mean survival time and resolved the liver injury when compared to the no-transplantation control animals. The transplanted hiPSC-EB-HLCs secreted human albumin into the host plasma throughout the examination period (2 weeks). Transplantation successfully bridged the animals through the critical period for survival after acute liver failure, providing promising clues of integration and full in vivo functionality of these cells after treatment with WIF-1 and DKK-1.

In other aspects, the invention is based, at least in part, on the discovery that human embryoid bodies (hEBs) can be generated using hiPSCs interlaced with endothelial cells. Advantageously, these embryoid bodies were generated without the need for rho-associated kinase inhibitors (ROCKi), and/or centrifugation (ROCKi/Spin-free). The four-stage hepatocyte differentiation protocol was applied to embryoid bodies generated with and without endothelial cells. The embryoid bodies were characterized for hepatic functionalities and markers in vitro and a D-galactosamine induced acute liver failure rat model was used for in vivo studies.

The differentiation of HLC was confirmed by the presence of gene expression and immunofluorescence of several hepatocyte markers such as Albumin, C-Met, CK-18, HNF-4α and several CYP450 isoforms. hiPSC+EC-EB-HLC showed increased amount of Albumin secretion in vitro compared to hiPSC-EB-HLC. hiPSC+EC-EB-HLC displayed lower secretion of Alpha-Fetoprotein compared to hiPSC-EB-HLC. Hepatocyte functions in vitro, such as Acetylated low-density lipoprotein uptake, Indocyanine green absorption and release after 6 hours, Glycogen storage, and cytoplasmic accumulation of neutral triglycerides and lipids were comparable between hiPSC+EC-EB-HLC and hiPSC-EB-HLC. The induction of several cytochromes P450 using different inducers demonstrated an increased activity of all the CYP450 tested for the hiPSC+EC-EB-HLC compared to hiPSC-EB-HLC. Differentiated cells displayed gene expression and secretion of all the intrinsic and extrinsic coagulation factors, showing the ability of both HLC and hEC to function as one organoid unit. Transplantation of hiPSC+EC-EB-HLC was associated with sustained rat serum human albumin at 14 days after transplant as compared to 3 days after transplantation among the hiPSC-EB-HLC group. Significantly, incorporation of hECs with hiPSCs in hEBs provided more sustained hepatocyte function in vivo after transplantation.

In other aspects, the invention is based, at least in part, on the discovery that HLCs with and without interlaced human endothelial cells are able to produce and secrete coagulation factors that are normally produced by both the primary hepatocyte and the endothelial cells in vivo. These coagulation factors include von Willebrand factor (vWF), Factor IX, Protein C, Factor X, Protein S, Factor V, Factor VIII, Antithrombin, Factor VII, Factor XI, C-reactive Protein, Factor XII, Prothrombin and Factor XIII. The HLCs with and without interlaced human endothelial cells are generated using the differentiation protocol disclosed herein. These HLCs allow for the treatment of patients suffering from blood coagulation disorders, such as hemophilia.

The HLCs with and without interlaced human endothelial cells are able to function in the Liver phase 2 detoxification pathway (i.e., Liver phase 2, also referred to as the conjugation pathway), whereby the liver cells add another substance (e.g., cysteine, glycine or a sulphur molecule) to a toxic chemical or drug to render it less harmful. This process also allows the toxin or drug to become water-soluble, so it can then be excreted from the body via watery fluids (e.g., bile or urine). This discovery will allow a potential cure for patients suffering from Crigler-Najjar Syndrome.

In another embodiment, the HLCs disclosed herein possess several activities of Cytochrome P450 enzymes, which are associated with the Liver phase 1 detoxification pathway. The Liver phase 1 pathway converts a toxic chemical into a less harmful chemical. This characteristic of the disclosed HLCs allows for the treatment of patients suffering from acute liver failure, and bridges these same patients to whole organ transplant. Thus, the disclosed HLCs, contrary to what happens the primary hepatocytes, not only do not lost their detoxification abilities over time, but instead the HLCs maintain and improve their capacity of metabolize toxins.

Accordingly, the invention provides cellular compositions comprising hepatocyte-like cells for transplantation that are produced in accordance with the methods of the invention, and methods of using such cells for the treatment of diseases characterized by a loss of liver function.

### Liver Disease and Dysfunction

Cellular compositions of the invention comprising hiPSC-EB-HLCs are useful for the treatment of any disease or disorder characterized by a loss of liver function. Such diseases include, but are not limited to patients suffering from liver failure, end-stage liver disease, cirrhosis, hepatitis B or C infection, hepatocellular carcinoma, Crigler-Najjar Syndrome, Urea Cycle Defects, Ornithine Transcarbamylase (OTC) Deficiency, Carbamoyl-Phosphate Synthetase I (CPS-1) Deficiency, Citrullinemia (Cit) disorder, Arginosuccinate Lyase (ASL) Deficiency, Familial Hypercholesterolemia, Hemophilia, Factor VII, Glycogen storage disease, Phenylketonuria (PKU), Infantile Refsum Disease, Progressive Familial Intrahepatic Cholestasis (PFIC-2), AlAT Deficiency, and Primary Oxalosis.

Patients with end-stage liver disease may be selected for treatment with a composition of the invention using any one or more of the following criteria: (a) histological (biopsy) diagnosis of cirrhosis or evidence of a nodular pattern of cirrhosis obtained by sonography, CT scan or MRI; (b) history of hepatic encephalopathy or clinical evidence of portal hypertension, such as esophageal varices or ascites; and (c) a Model for End Stage Liver Disease (MELD) Score of 15 to 24. These patients may have a patent portal vein with visualization of intrahepatic vessels. (Patients with transjugular intrahepatic portosystemic shunt (TIPSS) are eligible).

Patients with Non-Fulminant Liver Failure (Chronic Disease) may be selected for treatment with a composition of the invention using any one or more of the following criteria: a life expectancy of approximately 6 to 18 months. In some embodiments, the selected patient is ineligible for whole organ transplantation.

Patients with a diagnosis of non-resectable Hepatocellular Carcinoma staged as T3, according to the American Liver Study Group Modified Tumor-Node-Metastasis (TNM) Staging Classification, may optionally be treated prior to cell transplantation with TheraSphere® Ablation of the tumor(s). See “A Humanitarian Device Exemption Use Protocol of TheraShere® For Treatment of Unresectable Hepatocellular Carcinoma”, VCUMC, IRB # HM 10046.)

Patients with Fulminant Liver Failure (Acute/Fulminant Disease) may be treated with method of the invention.

Optionally, tacrolimus is prescribed to patients prior to transplantation at a dose of 0.5 mg/kg/day to be taken in two divided doses (i.e., 0.25 mg/kg) administered 12 hours apart beginning on the morning of Day-2, continuing on Day-1. The second dose of tacrolimus on Day-1 should be administered as close to 6:00 μm as possible (to facilitate 12 hour trough blood level (8-12 ng/mL) determination in the hospital the next morning. (Note: If the use of tacrolimus is not appropriate for a particular patient, cyclosporine may be instead be prescribed at an initial does of 6 mg/kg/day in two divided doses, administered 12 hours apart. Subsequent monitoring of trough cyclosporine blood levels and dosage adjustments to maintain a trough blood level of 200-300 ng/mL is recommended to be implemented as for tacrolimus.)

### Hepatocyte-Like Cells for Transplantation

Cells for transplantation display the characteristics of true hepatocytes, although they are termed “hepatocyte-like cells.” An increasing number of studies have investigated hepatic differentiation of human embryonic stem cells (hESCs) or hiPSCs and have provided insights into differentiation strategies. These studies have, in general, reached the consensus that the differentiation yields and culture uniformity are subject to the effects of multiple variables in the culture, including the form of the hiPSCs to start with, the differentiation substrates, the induction schemes, and scalability of the protocol. Hepatic differentiation of hESCs or hiPSCs usually starts by one of three methods, i.e., embryoid bodies (EBs) that are subsequently plated on diverse substrates, differentiation on mouse embryonic fibroblasts feeder layers, or differentiation on adherent feeder-free cultures (Rambhatla, L. et al., Cell Transplant 12, 1-11 (2003); Schwartz, R. E. et al. Stem Cells Dev 14, 643-655 (2005); Hay, D. C. et al. Cloning Stem Cells 9, 51-62 (2007)). EBs are 3-dimensional (3-D) hiPSC cell aggregates that can differentiate into cells of all three germ layers (endoderm, ectoderm, and mesoderm).

Events in the in vitro lineage-specific differentiation process within the EBs recapitulate those seen in vivo in the developing embryo, which justifies the use of EBs as a model to simulate the in vivo differentiation of hPSCs under in vitro culture conditions (Bratt-Leal, A. M. et al., Biotechnol Prog 25, 43-51 (2009)). Differentiation protocols starting from EBs are more scalable due to their higher tolerated density of cells within the clusters and the ability to be maintained in a suspension culture. Previously described techniques to reproducibly generate embryoid bodies from hiPSCs or hESCs have used the xeno-factor, rho-associated kinase inhibitors (ROCKi), and/or centrifugation (Subramanian, K. et al., Stem Cells Dev 23, 124-131 (2014)). As reported herein, the invention provides for the robust scalable production of homogeneous and synchronous hEBs from singularized hPSCs using non-adhesive round-bottom hydrophilic microwell arrays and eliminating both ROCKi xeno-factor and/or centrifugation. This new technique has allowed us to produce hiPSC-derived synchronized hEBs in large quantities for direct differentiation into the desired cell lineages.

Embryonic liver development follows three phases characterized by the formation of the definitive endoderm (DE), hepatoblast expansion and proliferation, and differentiation of hepatoblasts into mature, functional hepatocytes. Hepatoblasts are bipotential stem cells capable of giving rise to both major lineages of the liver: hepatocytes and biliary epithelial cells (cholangiocytes) (Duncan, S. A. Dev Dyn 219, 131-142 (2000)). The Wnt and β-catenin demonstrate individual as well as junctional effects in controlling postnatal liver development (Han, S. et al., Stem Cells 29, 217-228 (2011)). Increased β-catenin translocation to the nucleus correlates with an increase in cell proliferation (Apte, U. et al. Am J Physiol Gastrointest Liver Physiol 292, G1578-G1585 (2007)), whereas the Wnt pathway is considered as the major regulator of polarity and cell fate specifications (Cadigan, K. M. et al., Genes Dev 11, 3286-3305 (1997)). The effect of the Wnt and β-catenin on liver embryogenesis follows a highly temporally regulated profile (Hoeflich, K. P. et al. Nature 406, 86-90 (2000); Monga, S. P. et al. Gastroenterology 124, 202-216 (2003)). When combined, the Wnt/β-catenin pathway plays an important role in the hepato-biliary differentiation toward hepatocytes (Nejak-Bowen, K. et al., Organogenesis 4, 92-99 (2008); McLin, V. A. et al., Development 134, 2207-2217 (2007)), whereas stabilization of the β-catenin alone leads to increased propensity toward cholangiocytes over hepatocytes (Decaens, T. et al. Hepatology 47, 247-258 (2008)). Through the Wnt/β-catenin inhibition, it is possible to promote progression to hepatocytes at the hepato-biliary differentiation stage.

During phase II of liver development, hepatoblasts or hepatic progenitors undergo expansion while maintaining their de-differentiated state. Commitment to a hepatic fate is regulated by an array of the liver-enriched transcriptional factors that are present during phase III (Darlington, G. J. et al., Curr Opin Genet Dev 5, 565-570 (1995); Odom, D. T. et al., Science 303, 1378-1381 (2004)). Current conventional differentiation protocols follow a stepwise process from the initial endoderm formation, passing through hepatic progenitor cell induction, toward a mature hepatic phenotype without taking into account the important role of Wnt/β-catenin inhibition. The soluble factors that are administered at different stages of differentiation include: Activin A for the endoderm formation, FGF family factors for the progenitor hepatic specification, with the addition of BMP4 in some cases, and finally Oncostatin M and HGF for the maturation step (Si-Tayeb, K. et al., Hepatology 51, 297-305 (2010); Chen, Y. F. et al., Hepatology 55, 1193-1203 (2012); Song, Z. et al., Cell Res 19, 1233-1242 (2009); Sullivan, G. J. et al., Hepatology 51, 329-335 (2010); Takata, A. et al. Hepatol Int 5, 890-898 (2011); Touboul, T. et al., Hepatology 51, 1754-1765 (2010)). Notable limitations with current protocols include low scalability, remnant immature genotypes after differentiation, and poor long-term cell functionality following transplantation (Wu, X. B. et al., Hepatobiliary Pancreat Dis Int 11, 360-371 (2012)).

Disclosed herein are compositions and methods that provide for three-dimensional multicellular spheroid culture-based hepatic differentiation protocol that starts from hEBs and employs two inhibitors of the Wnt/β-catenin pathway to mimic the differentiation stage during hepatogenesis in vivo. The scalability of the in vitro hepatic differentiation protocol allows the production of human hepatocytes in large quantities for transplantation therapy. The functionality of hiPSC-derived HLCs was characterized in an animal model of acute liver failure.

### Selection of Cells for Transplantation

Once a patient is selected as a candidate for transplantation, cells to be used for transplantation must be selected. Several criteria are disclosed herein for the selection of compatible cells for hepatocyte transplantation. Such criteria include the availability of recipient-specific compatible cells for transplantation. In one embodiment, compatible cells are those from an ABO-compatible donor with no HLA Class I antigen to which the recipient has preformed antibodies. Cells from blood type O donors (“universal donors”) may be given to patients with blood type A, B, AB, and O. Hepatocytes from an EBV-positive or CMV-positive donor may be administered to EBV-negative or CMV-negative recipients if the recipient can receive standard Transplant Center whole organ CMV/EBV prophylaxis. See Tables 1 and 2 below:

TABLE 1
CMV Infection Prophylaxis

| CMV | | | Length of |
|---|---|---|---|
| Prophylaxis | Dose | Start | Treatment |
| Ganciclovir | IV, 2.5 mg/kg/day | Immediate post | 7-14 days |
| | (with dose adjustment | Hepatocyte | |
| | for renal dysfunction | Infusion | |
| Acyclovir | Adult: Oral, 800 mg, | At conclusion of | 3 months |
| | four times daily | Ganciclovir | |
| | Pediatric: Oral, | Therapy | |
| | 20 mg/kg/dose, | | |
| | four times daily | | |
| | (with dose adjustment | | |
| | for renal dysfunction) | | |

TABLE 2
EBV Infection Prophylaxis

| | | | Length of |
|---|---|---|---|
| EBV Prophylaxis | Dose | Start | Treatment |
| Acyclovir | Adult: Oral, 800 mg, | At conclusion of | 3 months |
| | four times daily | Ganciclovir therapy | |
| | Pediatric: Oral, | | |
| | 20 mg/kg/dose, | | |
| | Four times daily | | |
| | (With dose | | |
| | adjustment for renal | | |
| | dysfunction) | | |

Regarding the dose scheme and rationale, hepatocyte numbers used for transplantation are determined by the total number of hepatocytes available that matches the blood type for each patient. Optimally, hepatocytes from a single donor are recommended to be used for each patient.

### Characterization of Hepatocyte-Like Cells

The invention provides methods for growing large numbers of hepatocyte-like cells in vitro. Cells produced according to the methods of the invention are characterized or monitored for the expression of markers that identify them as mature hepatocyte-like cells. In one embodiment, cells of the invention are characterized (e.g., using immunohistochemistry) for the expression of SOX17 and FOXA2, which are markers for the endodermal stage; HHEX and GATA4, which are markers for the foregut endoderm; AFP and HNF-4a, which are markers for hepatic progenitor cells; and ALBUMIN and CK-18, which are markers for mature hepatocyte-like cells.

In other embodiments, cells of the invention are characterized for secretion of albumin, fibrinogen and alpha fetoprotein (AFP) into the medium. In other embodiments, cells of the invention are characterized for the intracellular concentration of urea, which is detected after differentiation. In other embodiments, cells of the invention are characterized for indocyanine green uptake and/or release; cytoplasmic accumulation of neutral triglycerides and lipids (e.g., LDL) as measured, for example, by Oil-Red O staining; glycogen storage as measured, for example, by PAS staining, acetylated low-density lipoprotein (DiI-ac-LDL) uptake; cytochromes P450 enzyme activity as measured, for example, by incubating the cells with different inducers: Omeprazole for CYP1A2, Rifampicin for CYP3A4 and Phenobarbital for CYP2B6; and metabolized ammonia. In other embodiments, the cells of the invention presented liver phase II functions after differentiation. Liver phase II represent the conjugation of metabolites for their final excretion in the urine. In some embodiments, the differentiated clusters are suitable for use as a therapeutic agent for Crigler-Najjar Syndrome, cirrhosis, hepatitis B or C infection, hepatocellular carcinoma, Crigler-Najjar Syndrome, Urea Cycle Defects, Ornithine Transcarbamylase (OTC) Deficiency, Carbamoyl-Phosphate Synthetase I (CPS-1) Deficiency, Citrullinemia (Cit) disorder, Arginosuccinate Lyase (ASL) Deficiency, Familial Hypercholesterolemia, Hemophilia, Factor VII, Glycogen storage disease, Phenylketonuria (PKU), Infantile Refsum Disease, Progressive Familial Intrahepatic Cholestasis (PFIC-2), AlAT Deficiency, and Primary Oxalosis.

### Delivery of Hepatocyte-Like Cells

In one embodiment, Hepatocyte-like Cells of the invention are delivered by Portal Vein Infusion. The dosage of cells delivered will be determined by a clinician based on the individual needs of the patient. In one embodiment, 10-200 (e.g., 5, 10, 20, 30, 40, 50, 60, 70, 80, 90, 100, 125, 150, 175, 200)×10⁶ hepatocytes per kilogram of patient body weight is delivered at an infusion rate of 5-10 ml/kg/hr and a concentration of 1-10×10⁶ hepatocytes/l ml. In one embodiment, hepatocyte-like cells are suspended in Dextrose 5% in Lactated Ringers Solution (D5LR). In one embodiment, infusion is carried over 30 minute intervals, and the cell mixture should be kept on wet ice to maintain a mild hypothermic 32 degree solution temperature.

In one embodiment, Hepatocyte-like Cells of the invention are delivered by Splenic Artery Infusion. In one embodiment, no more than 6×10⁸ hepatocytes are delivered per infusion. Using the same speed, concentration and temperature directives as the portal vein infusion, which is, an infusion rate of 5-10 ml/kg/hr and a concentration of 1-10×10⁶ hepatocytes/1 ml, hepatocytes suspended in D5LR. In one embodiment, infusion is carried over 30 minute intervals, and the cell mixture should be kept on wet ice to maintain a mild hypothermic 32 degree solution temperature.

Several routes of hepatocyte administration are disclosed herein. Hepatocytes may be transplanted via the intraportal or intrasplenic routes. The site of infusion should be chosen such that it offers the maximum potential for patient safety, successful hepatocyte engraftment, function and viability. Specific factors to consider for route administration include: (a) the etiology of the liver disease; (b) portal vein, splenic vein and splenic artery patency; (c) evidence of portal hypertension; (d) relative normalcy of the liver's architecture; (e) stage of cirrhosis; (f) liver size; (g) spleen Size; (h) age and size of patient; (i) prior intraperitoneal surgery; and (j) patient's functional status.

Several guidelines are disclosed herein for cell transplantation and clinical preparation: (a) patients should be admitted into the hospital on the afternoon of Day-1 or just after midnight on Day 1; (b) patients should have an evening dose of tacrolimus administered. Prior to transplant, a clinician may administer any one or more of the following medications: Fluconazole, 400 mg, Famotidine, 20 mg, Methylprednisolone, 250 gm. Fulminant Liver Failure patients may be administered Vitamin K as needed; Ranitidine, broad spectrum antibiotics N-acetyl-cystein.

Several criteria are disclosed herein for hepatocyte transplantation and interventional radiology. Transplantation may take place in an interventional radiology suite. In one embodiment, the patient may be administered the appropriate dose of antibiotic Piperacillin/Tazobactum (Zosyn) one hour prior to catheterization with a second dose to be administered in 6 hours. An appropriate substitution may be made in the event of patient history of allergy. Patients should be continuously monitored for blood pressure, heart rate, respiratory rate, oxygen saturation throughout the catheterization process. The following medications may be administered to induce conscious sedation: (i) 50 mg Diphenhydramine, IV, administered at a rate of 25 mg/minute; (ii) 50 μg Fentanyl, IV, infused over 3-5 minutes; (iii) 1.0 to 1.5 mg Midazolam, every 2 minutes until desired level of sedation is achieved.

Under sterile conditions, hepatocytes are infused into the splenic artery or portal vein via a 4 or 6-french angiographic catheter advanced from a percutaneous sheath into the femoral artery or transhepatic route under fluoroscopic guidance. Vessel patency may be confirmed twice with contrast dye (Visipaque®) injection or equivalent. Monitoring should take place routinely, with documentation at least every 15 minutes, of airway pressures, intracranial pressures (where indicated), cardiac monitoring, blood pressure, heart rate, respirations and oxygen saturation throughout the infusion procedure.

The catheter can be removed after completion of the infusion (and flush) of the liver cells and the final post-infusion assessment of vessel patency. Immediately before the catheter is withdrawn, to reduce the chances of bleeding, a Gelform plug and/or collagen/thrombin paste may be used to embolize the entire peripheral catheter tract. The patient should be monitored for a minimum of two hours in a radiology suite after the procedure. Discharge from a Interventional Radiology Department to a General Clinical Research Center or appropriate unit (ICU) may occur after the patient is assessed to be alert, oriented and maintaining stable vital signs and respiratory function.

Regular assessment of vital signs, level of alertness and site of catheterization should occur after the procedure. Documentation of findings should be recorded every 15 minutes, post procedure for one hour, every 30 minutes for two hours, every one hour for four hours and then every two hours until discharge. It is recommended to repeat antibiotic dosing six hours after initial dose given pre-procedure. The clinician may administer any one or more of the following medications after transplantation: tacrolimus, and prednisone.

### Combination Therapies

Optionally, a cellular composition of the invention may be administered in combination with any therapy that is conventionally used for the treatment of a liver disease. In certain embodiments, tacrolimus, cyclosporine, ganciclovir, and/or acyclovir are administered prior to, concurrent with, or subsequent to administration of a cellular composition of the invention. In another embodiment, patients with Hepatitis B virus are treated with lamivudine, truvada, or best antiviral/individual pt. and/or Hepatitis B immune globulin.

### Cellular Compositions

Compositions of the invention include pharmaceutical compositions comprising hepatocyte-like cells, or their progenitors, and optionally endothelial cells, and a pharmaceutically acceptable carrier. In some embodiments, the endothelial cells are interlaced with the hiPSC before the differentiation process to obtain HLCs. Administration can be autologous or heterologous. For example, can be obtained from one subject, and administered to the same subject or a different, compatible subject.

Hepatocyte-like cells can be administered via localized injection, including catheter administration. In particular embodiments, a cellular composition is administered via portal vein, splenic vein or splenic artery. When administering a therapeutic composition of the present invention (e.g., a pharmaceutical composition), it will generally be formulated in a unit dosage injectable form.

Cellular compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

One consideration concerning the therapeutic use of hepatocyte-like cells of the invention is the quantity of cells necessary to achieve an optimal effect. The quantity of cells to be administered will vary for the subject being treated. In a preferred embodiment, between 10⁴ to 10⁸, more preferably 10⁵ to 10⁷, and still more preferably, 3×10⁷ hepatocyte-like cells of the invention can be administered to a human subject.

Hepatocyte-like cells of the invention can comprise a purified population of cells that express markers and have functional activities consistent with mature hepatocytes. Those skilled in the art can readily determine the percentage of hepatocyte-like cells in a population using various well-known methods, such as fluorescence activated cell sorting (FACS). Preferable ranges of purity in populations comprising hepatocyte-like cells are about 70 to about 75%, about 75 to about 80%, about 80 to about 85%; and still more preferably the purity is about 85 to about 90%, about 90 to about 95%, and about 95 to about 100%. Purity of hepatocyte-like cells can be determined according to the marker profile within a population. Dosages can be readily adjusted by those skilled in the art (e.g., a decrease in purity may require an increase in dosage).

The skilled artisan can readily determine the amount of cells and optional additives, vehicles, and/or carrier in compositions and to be administered in methods of the invention. Typically, any additives (in addition to the active hepatocyte-like cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Kits

Hepatocyte-like cells of the invention may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Examples

### Example 1: Differentiation of hiPSC Embryoid Bodies (hiPSC-EBs) in 3D Culture into Hepatocyte-Like Cells (HLCs)

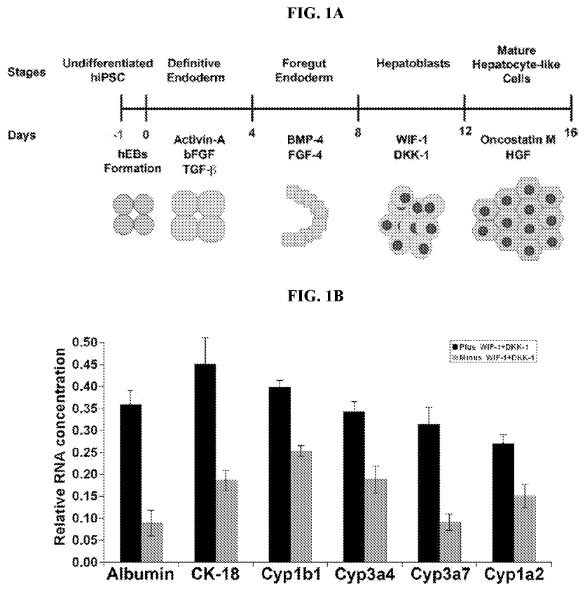

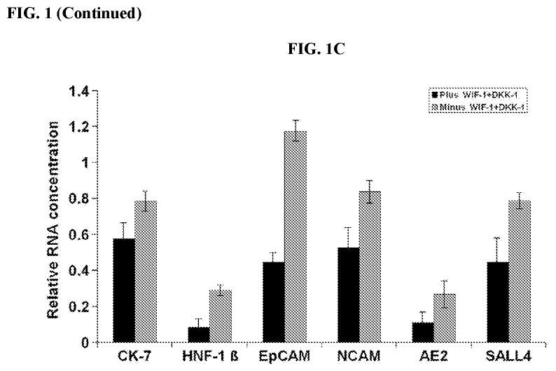

Embryoid bodies were produced reliably and efficiently with high viability using agarose micro-well arrays and Teflon stamps. An 80% confluent six-well plate containing 1.2×10⁶ dissociated hiPSC produced approximately 280 embryoid bodies (FIG. 7). The hiPSC-EBs underwent a 4-stage hepatic differentiation process in a continuous 3D culture. The differentiation protocol recapitulates the developmental stages that occur during embryogenesis in vivo (FIG. 1A). Starting from pluripotent stem cells (PS), the four stages were definitive endoderm (DE), foregut endoderm (FE), hepatic progenitor cells or hepatoblast (HPC) and mature hepatocytes (MH). Each stage of the differentiation protocol lasted 4 days with two every-other-day medium changes. The protocol used two novel factors for Wnt inhibition: Wnt inhibitory factor 1 (WIF-1) and Dickkopf-1 (DKK-1) at the HPC stage. The working concentrations of WIF-1 and DKK-1 followed the suggested manufacturer ranges and literature review of studies in mouse and human cell lines. The concentrations that showed the maximum effect in WNT inhibition in those studies were used.

To evaluate the effect of the two Wnt inhibitors on hepatic differentiation and ensure the reproducibility of our results, all the experiments were performed in parallel starting from the same batch of hiPSC-EBs for both the treated and non-treated control (with and without inhibitors), as well as the undifferentiated hiPSC-EBs and adult human hepatocyte (negative and positive controls).

At the end of the differentiation process, semi-quantitative PCR was used to analyze markers for cholangiocyte and hepatocyte-specific gene expression. The presence of the two Wnt inhibitors in the differentiation protocol resulted in increased propensity of the differentiating hiPSC-EBs toward the two different lineages. The hiPSC-EBs differentiated with both WIF-1 and DKK-1 exhibited much higher expression of hepatocyte-specific markers relative to the ones differentiated without the two Wnt inhibitors (FIG. 1B). The hiPSC-EBs differentiated without WIF-1 and DKK-1 demonstrated greater expression of cholangiocyte-specific markers (FIG. 1C). The differences between the conditions with and without WIF-1 and DKK-1 in gene expression were all statistically significant (p<0.0001).

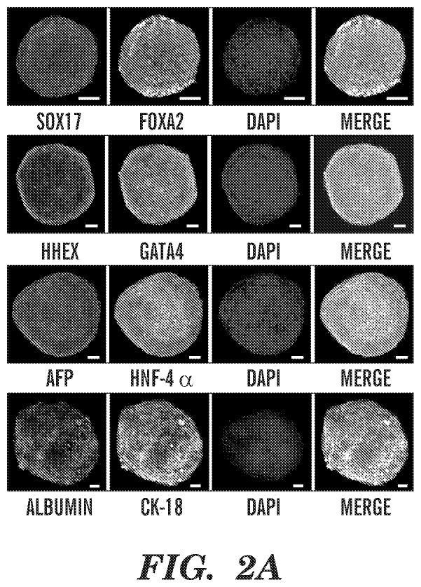

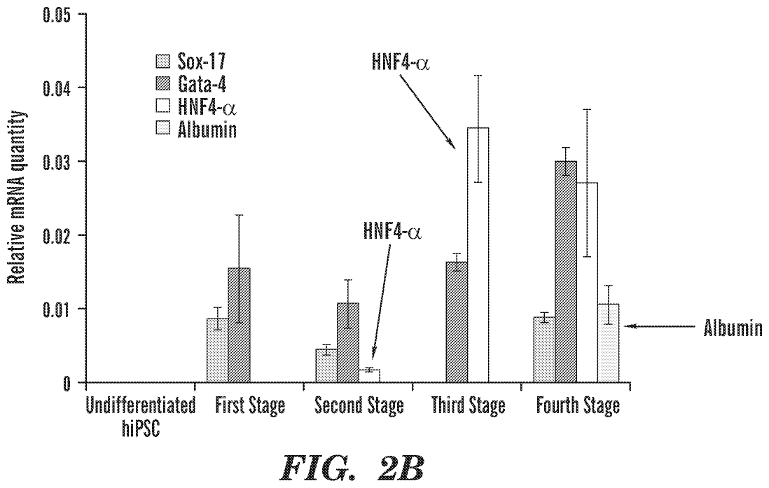

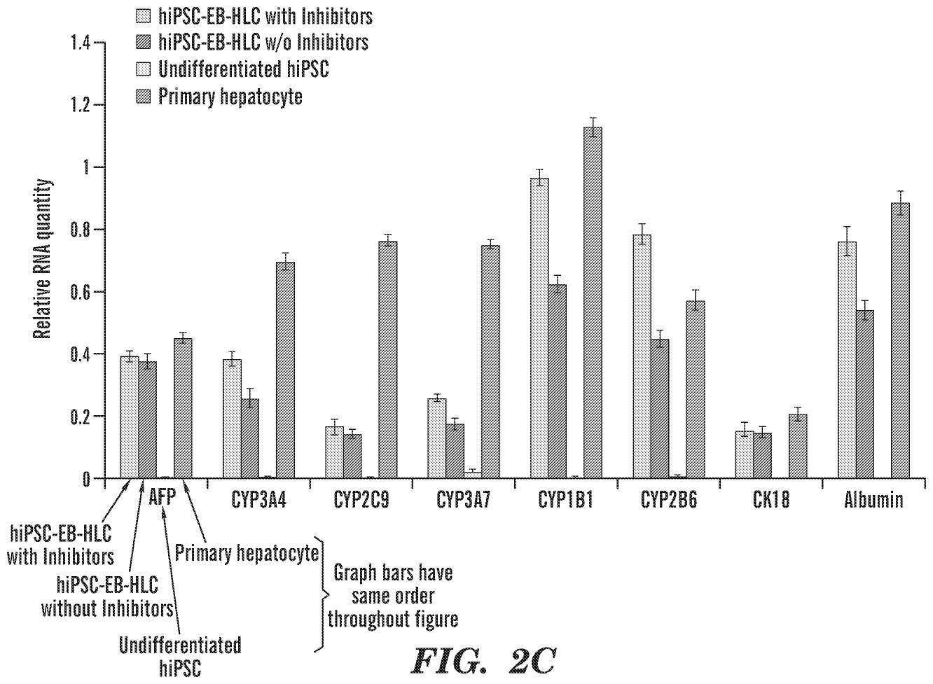

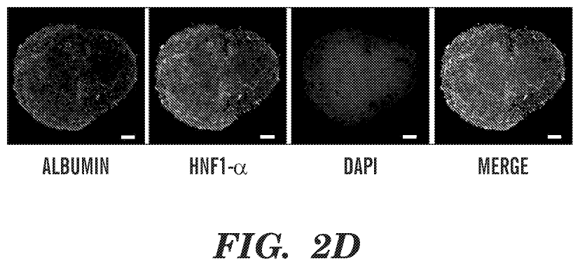

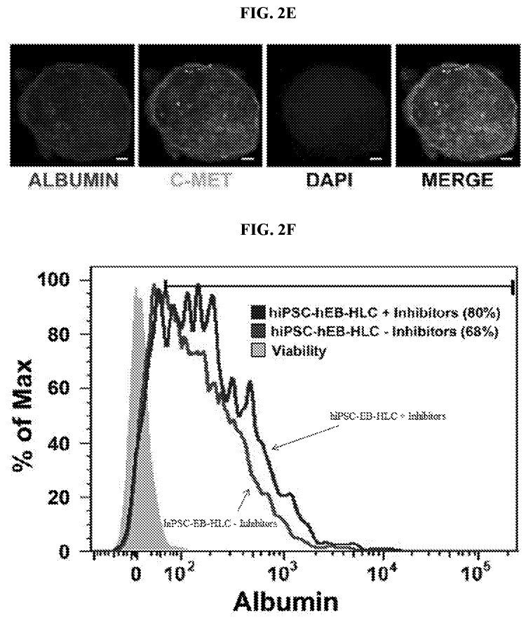

To ensure the stepwise differentiation of the hiPSC-EBs using the protocol of this example, stage-specific protein analyses were performed at the end of individual stages. The differentiating hiPSC-EBs exhibited a temporal regulated pattern of stage-specific intracellular hepatic protein expression at the end of each individual stage, including FOXA2 and SOX17 at the end of definitive endoderm, HHEX and GATA-4 at the end of foregut endoderm, HNF-4α and AFP at the end of hepatic endoderm, and Albumin and CK-18 at the end of mature hepatocyte stage (FIG. 2A).

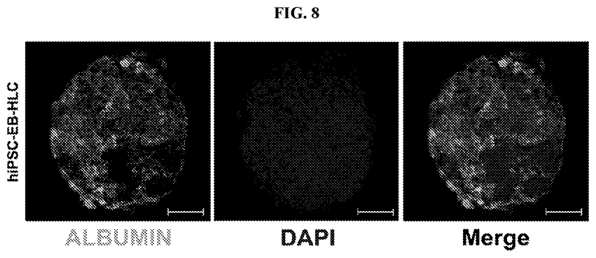

To determine the gene expression profile of the differentiating hiPSC-EBs, quantitative RT-PCR (qRT-PCR) was used at various time points during the differentiation protocol to measure the relative quantities of stage-specific genes at the mRNA level (FIG. 2B). Undifferentiated hiPSCs were used as negative controls. The mRNA of the undifferentiated hiPSCs was negative for markers of all four differentiation stages. In general, expressions of stage-specific genes by differentiating hiPSC-EBs peaked at the respective stages and gradually declined subsequent to that stage. The only exception was GATA-4, a marker for stage II (foregut endoderm), which was induced as early as stage I and peaked at stage IV. At stage IV, albumin mRNA expression was seen. Quantitative RT-PCR for both conditions, with and without WIF-1 and DKK-1, displayed the presence of mRNA for five P450 isoforms (Cyp1B1, Cyp2C9, Cyp3A4, Cyp2B6 and Cyp3A7), Alpha fetoprotein, Albumin, and CK18 in the terminally differentiated hiPSC-EB-HLCs (FIG. 2C). In particular, hiPSC-EBs treated with the protocol containing WIF-1 and DKK-1 showed a higher expression pattern for all the markers compared to the hiPSC-EBs treated with the protocol without the two inhibitors. Undifferentiated hiPSCs were used as negative control and human primary hepatocytes were used as positive control. Following the differentiation program, terminally differentiated hiPSC-EB-HLCs expressed a repertoire of mature hepatocyte-specific proteins, as evidenced by immunohistochemical co-staining of ALBUMIN and HNF-1α, and ALBUMIN and C-MET (FIG. 2D, FIG. 2E). The comparison for the hepatic differentiation yield between the two protocols with and without WIF-1 and DKK-1 was evaluated by FACS analysis using the albumin-positive cells as the reference marker. The results showed an increased yield of Albumin positive cells in the hiPSC-EBs treated with the protocol with the two inhibitors compared with the one without inhibitors (80% vs 68% respectively) (FIG. 2F). A confirmation of this result for the protocol with both inhibitors was obtained by counting under confocal microscope the fraction of albumin-positive cells in each optical section averaged over a minimum of 10 microscopic fields for each cluster with a minimum of 50 different clusters per differentiation condition. The differentiation protocols of this example, with both Wnt inhibitors WIF-1 and DKK-1, had consistently yielded an over 80% high-purity hepatocytes population when compared to a 70% hepatic differentiation yield that is usually seen with other differentiation protocols. FIG. 8 shows representative images used for cell counting to determine the hepatic differentiation yield.

### Example 2: hiPSC-EB-HLCs Displayed Morphology and In Vitro Functional Hepatic Characteristics

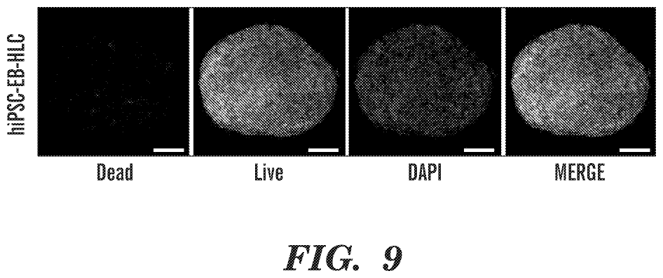

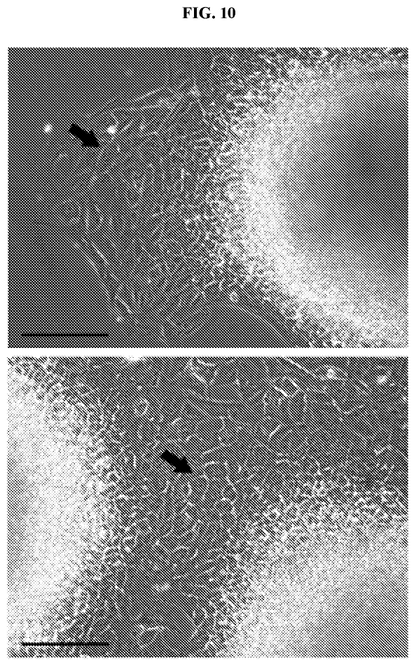

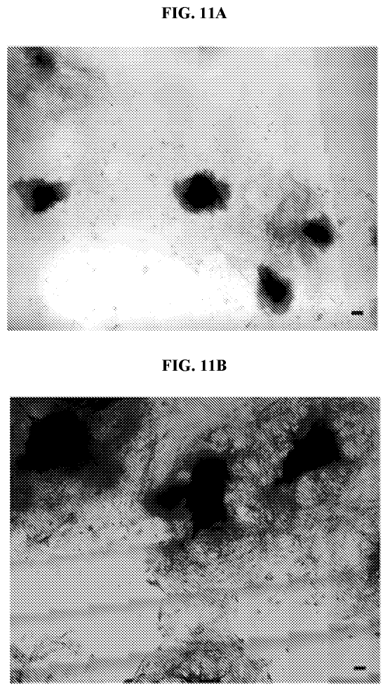

Morphological assessment of hiPSC-EBs undergoing differentiation in 3D culture revealed a progressive increase in cluster size from approximately 500 μm to 800-1,000 μm by the end of differentiation without any core necrosis at any time (FIG. 9). In order to further assess the cellular morphology at the end of the differentiation protocol, the hiPSC-EB-HLCs in 3D culture were placed in a Matrigel-coated plate and allowed to adhere. Over the course of 1 week, the hiPSC-EB-HLCs adhered to the surface of the plate and began to spread in a monolayer. Light microscopy showed that hiPSC-EB-HLCs were morphologically polygonal with enriched cytoplasmic granules (FIG. 10), replicating the morphological features of polygonal, vacuolated primary human hepatocytes. This result was observed for both conditions studied.

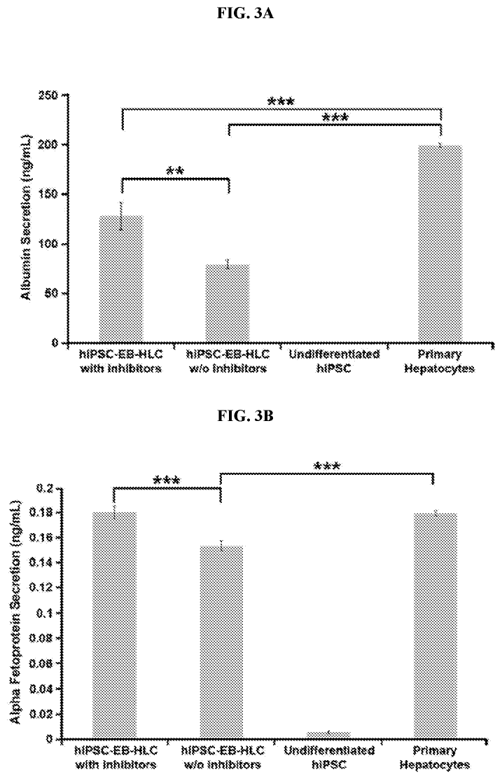

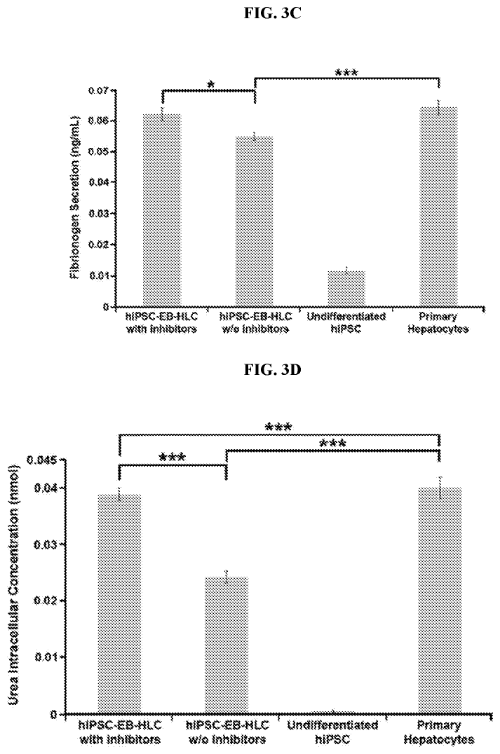

Examination of the conditioned culture medium indicated secretion of hepatic proteins by the hiPSC-EB-HLCs 48 hours following the completion of the differentiation process with our protocol with and without WIF-1 and DKK-1. hiPSC-EB-HLCs showed a different secretion pattern for all the proteins examined between the conditions with and without inhibitors, with increased secretion with the inhibitors. In particular, the condition with the inhibitors demonstrated increased albumin secretion (120-130 ng/ml vs. 76-80 ng/ml for 5×10⁵ cells respectively; p=0.0046). This corresponded approximately to 60% and 40% respectively of albumin production by primary human hepatocytes (128 ng/ml vs. 199 ng/ml, p<0.0009; 80 ng/ml vs. 199 ng/ml, p<0.0001). The condition with the two inhibitors showed statistically significantly higher secretion of both alpha fetoprotein (AFP) (0.18 ng/ml vs. 0.15 ng/ml, p=0.0007), and fibrinogen (0.062 ng/ml vs. 0.055 ng/ml, p=0.0175) relative to the condition without the inhibitors. Both AFP and fibrinogen in the conditions with WIF-1 and DKK-1 showed total protein concentration at levels that were equivalent to those of primary human hepatocytes (AFP: 0.18 ng/ml vs. 0.19 ng/ml, p=0.69; Fibrinogen: 0.062 vs. 0.064, p=0.0015) (FIG. 3A, FIG. 3B, FIG. 3C). In addition, the hiPSC-EB-HLCs under the condition with the inhibitors demonstrated an intracellular urea concentration that was statistically significantly higher relative to that of the hiPSC-EB-HLCs under the condition without the inhibitors (0.0388 nmol vs. 0.024 nmol, p<0.0001) (FIG. 3D). Undifferentiated hiPSCs were used as negative control, in which production of the proteins was absent at all times (p<0.01) (FIG. 3).

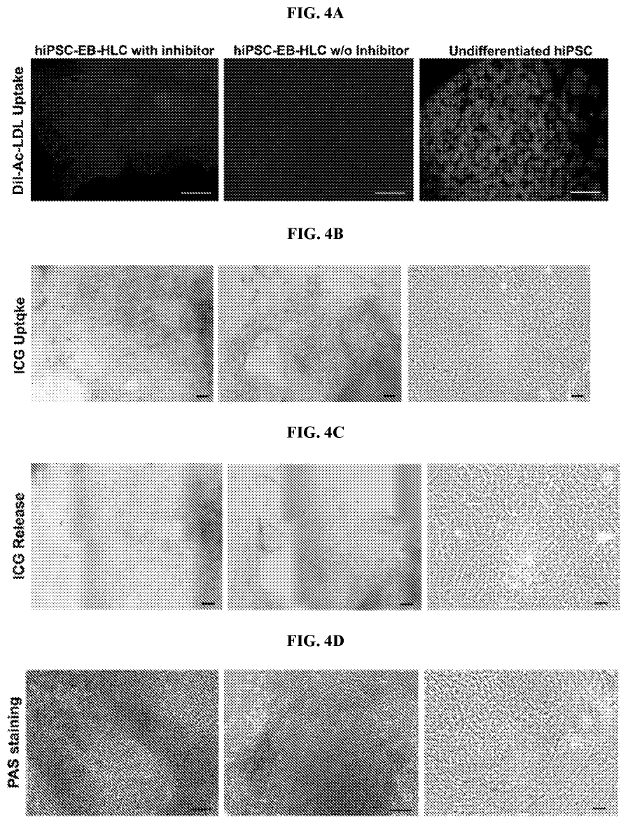

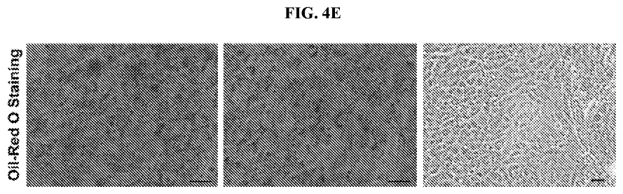

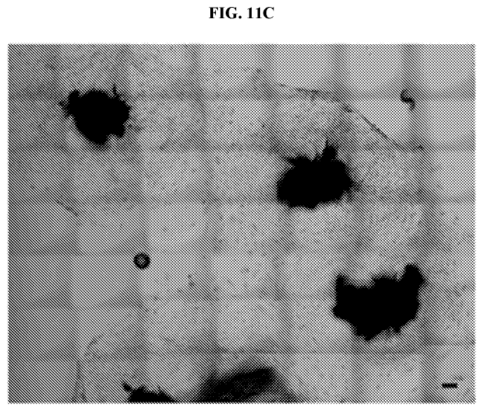

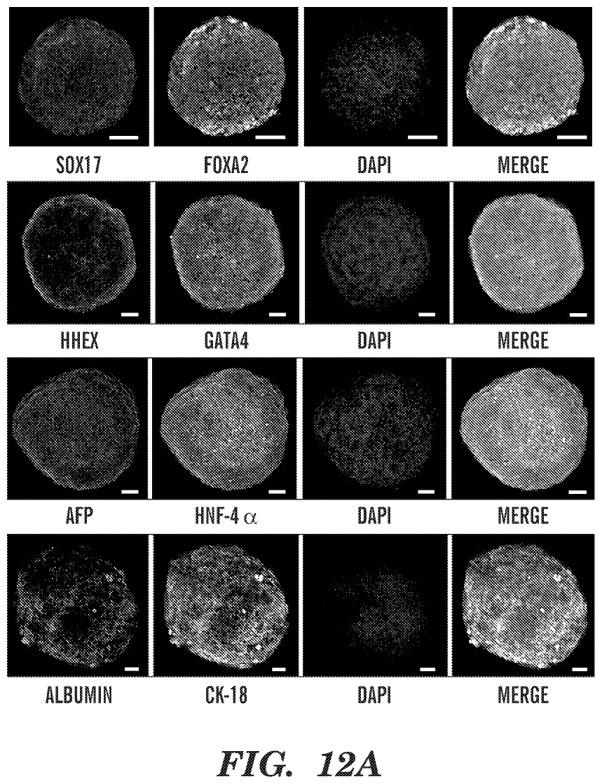

To assess the functional activities of the hiPSC-EB-HLCs, differentiated hiPSC-EB-HLCs in 3D culture were placed in a Matrigel-coated plate and allowed to adhere and spread in a monolayer (FIG. 11A, 11B, 11C). The hiPSC-EB-HLCs of both conditions with and without WIF-1 and DKK-1 displayed similar functional activities typical of mature primary hepatocytes, such as acetylated low-density lipoprotein (DiI-ac-LDL) uptake (FIG. 4A), indocyanine green (ICG—Cardiogreen) absorption and release after 6 hours (FIG. 4B, FIG. 4C), glycogen storage (FIG. 4D), and cytoplasmic accumulation of neutral triglycerides and lipids (FIG. 4E). Undifferentiated hiPSCs were used as negative control (right panel of FIG. 4) and did not demonstrate any of the activities above.

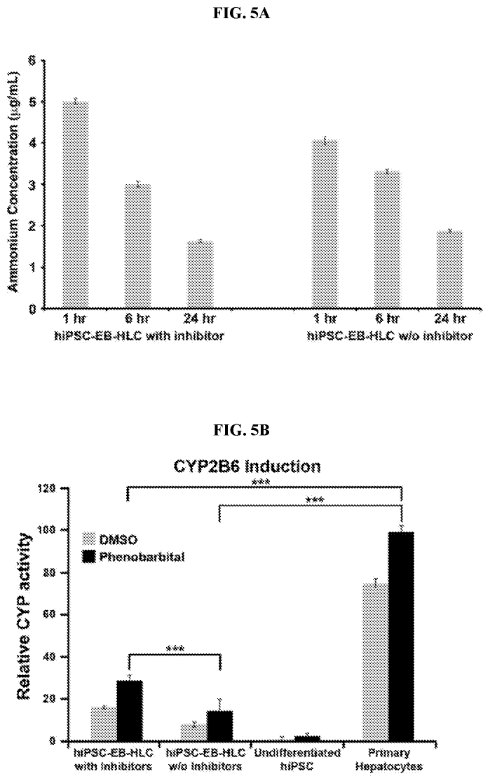

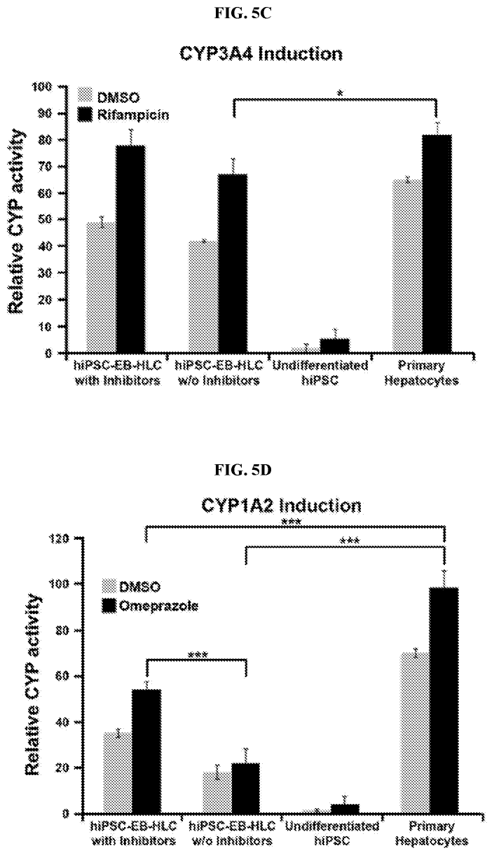

Ammonia metabolism via the urea cycle is an essential function of hepatocytes. Ammonia metabolism was evaluated by changes in ammonium concentration in the cell culture supernatant for both experimental conditions over a 24-hour period after addition of ammonium chloride of known concentration. Ammonium chloride standard of 1 mM was added to culture dishes containing 100 differentiated hEBs in suspension deriving from hiPSC differentiated with the protocol with and without WIF-1 and DKK-1. Supernatant was collected and ammonium concentration was measured at 1-, 6- and 24-hour intervals after ammonium chloride addition. There was a steady decrease in ammonium concentration in the supernatant over a 24-hour period for both conditions (FIG. 5A). In particular, there was not a statistically significant decrease in ammonium concentration between the sample treated with the two inhibitors and the one without. However, the levels of ammonium chloride at 24 hours showed a higher percentage of loaded ammonium that was metabolized by the hiPSC-EB-HLCs with the two inhibitors compared to the cells treated without inhibitors (70.15±5.12% vs. 60.32±3.25% respectively.

Next the detoxification abilities of the hiPSC-EB-HLCs were examined in vitro by characterizing the activities of Cytochrome P450 (CYP450) enzymes, the major hepatic enzymes that perform detoxification. Three CYP isoforms were tested by measuring the increase in CYP isoform gene expression in response to exposure to their respective inducers for 72 hours. The three inducers and CYP isoforms were Omeprazole (CYP1A2), Phenobarbital (CYP2B6), and Rifampicin (CYP3A4). DMSO was used as a control in cell co-culture to test the basal activity of the different CYP450. The results of this example indicated significant increases in the activities of all the tested isoforms of CYP450 in cell culture relative to the DMSO control (FIG. 5B, FIG. 5C, FIG. 5D). The hiPSC-EB-HLCs treated with the protocol containing WIF-1 and DKK-1 displayed increased CPY expression when compared to the one without inhibitors, in response to Phenobarbital (28.16±2.58% vs. 14.23±3.48%, p=0.001), Rifampicin (78.51±6.82% vs. 67.31±5.73%, p=0.062), and Omeprazole (54.26±4.21% vs. 22.12±2.34%, p=0.0002). Following induction, the hiPSC-EB-HLCs treated with the two inhibitors displayed similar CYP activity relative to primary hepatocytes for CYP3A4 (78 vs. 82, p=0.417), but statistically significantly lower CYP activities for CYP2B6 and CYP1A2 relative to primary hepatocytes (28 vs. 98, p<0.0001, and 54 vs. 98, p=0.0007, respectively). In comparison, hiPSC-EB-HLCs treated without the two inhibitors had all statistically significantly lower CYP activities for all the isoforms when compared with primary hepatocytes (CYP3A4: 67 vs. 82, p=0.0232; CYP2B6: 14 vs. 98, p<0.0001; CYP1A2: 22 vs. 98, p<0.0001). Undifferentiated hiPSC-EBs did not demonstrate any activities of any of the tested isoforms of CYP450.

### Example 3: Transplantation of hiPSC-EB-HLCs Resulted in Prolonged Survival and Human Albumin Release

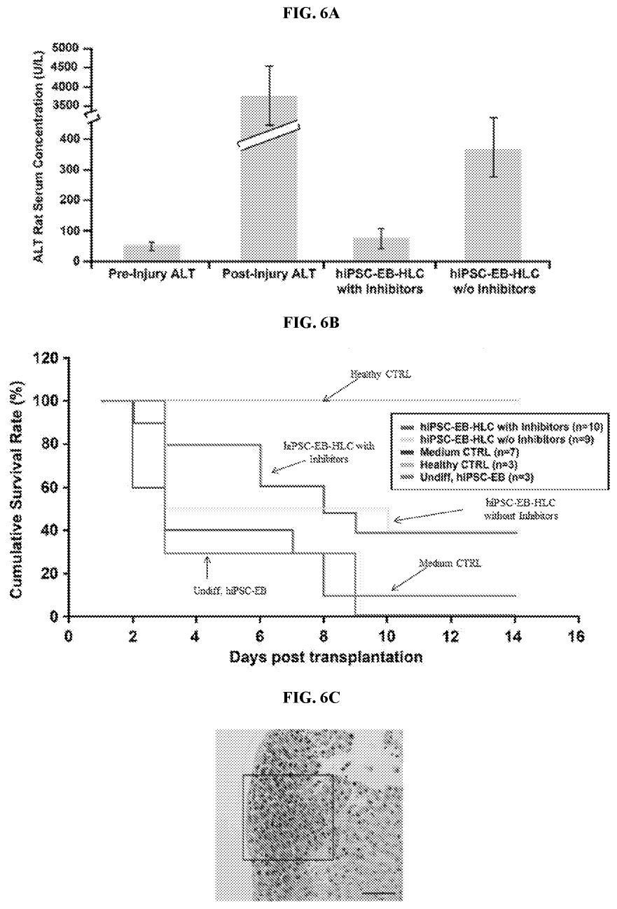

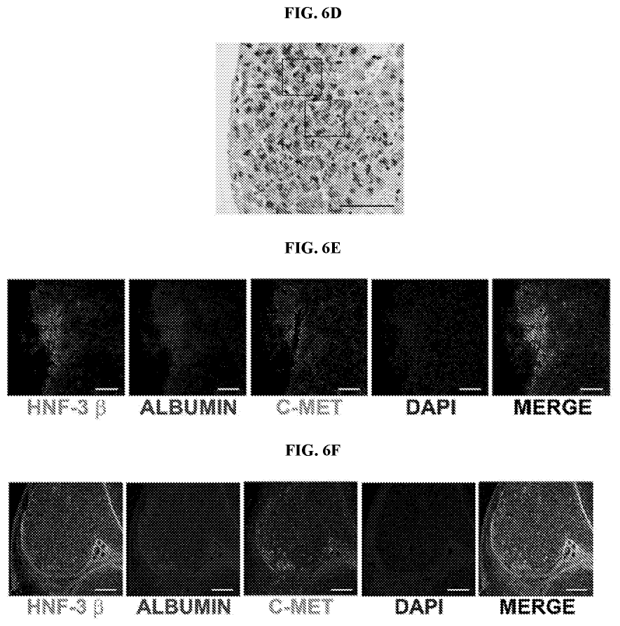

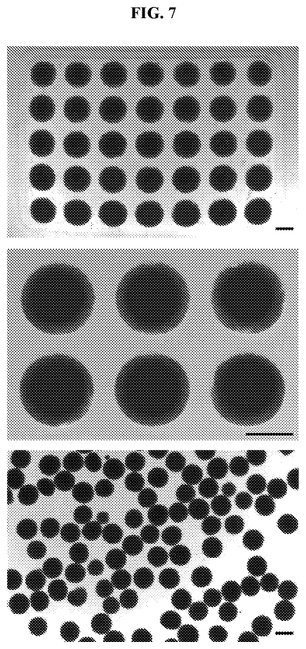

The d-galactosamine-induced model of acute liver failure in rats resulted in widespread hepatic necrosis within 24 to 48 hours after injury. Deaths occurred as early as 2 to 3 days after induction of liver failure and nearly 100% mortality was reached within 9 to 10 days after induction. Cell transplantation was performed 14 to 16 hours after induction of liver injury. Alanine aminotransferase (ALT) was used as a marker of liver injury. The mean ALT value (3781 U/L) increased significantly relative to the pre-injury condition (53 U/L), which then normalized to 78 U/L following transplantation of hiPSC-EB-HLCs treated with the inhibitors and 364 U/L for the hiPSC-EB-HLCs without inhibitors, indicating resolution of lethal liver injury for both experimental conditions (FIG. 6A).

The Kaplan-Meier survivals were determined for 14 days after cell transplantation. Almost all the no-cell medium control animals and the animals receiving undifferentiated hiPSC-EBs died within 5 to 8 days after the induction of liver failure (FIG. 6B). Through the examination period (14 days), animals receiving the hiPSC-EB-HLCs treated with the inhibitors trended towards higher mean survival (FIG. 6B) compared to the ones receiving the hiPSC-EB-HLCs without inhibitors (9.0±4.76 vs. 8.33±5.98 p=0.7902) (Table 1).

TABLE 1

| | | Mean | 72 hrs Rat serum- | 14 days Rat serum- | Human |
|---|---|---|---|---|---|
| | Survival to 14 days | survival | Human albumin | Human albumin | albumin |
| hiPSC-EB-HLC | 40.0% (4/10) | 9.0 ± 4.76 | 1.63 ± 0.43 | 28.20 ± 7.8 | 80.0% |
| with inhibitors (n = | | | ng/ml | ng/ml | (8/10) |
| 10) | | | | | |
| hiPSC-EB-HLC | 38.6% (4/9) | 8.33 ± | 0.20 ± 0.05 | 18.80 ± 5.4 | 66.0% (6/9) |
| w/o inhibitors (n = | | 5.98 | ng/ml | ng/ml | |
| 9) | | | | | |
| Undifferentiated | 33.3% (1/3) | 8.7 ± 5.5 | 0 | 0 | 0% (0/3) |
| iPSC EB (n = 3) | | | | | |
| Healthy Control (n = 3) | 100% (3/3) | 14 | 0 | 0 | 0% (0/3) |
| Negative Control | 14.3% (1/7) | 5.4 ± 4.5 | 0 | 0 | 0% (0/7) |
| (media only) | | | | | |

Table 1. shows that up to 14 days post-transplantation, there was a trend towards longer mean survival of animals receiving hiPSC-EB-HLCs treated with inhibitors relative to the hiPSC-EB-HLCs without inhibitors, but did not reach statistical significance (9.0 vs. 8.33 days, p=0.7902). At both time points of examination, i.e., 72 hrs and 14 days post-transplantation, human albumin was detected in the serum of the survived animals receiving hiPSC-EB-HLCs treated with the two inhibitors in a greater amount when compared with the ones receiving the hiPSC-EB-HLCs without inhibitors. Human albumin was not detected in the serum of any of the control animals at any time.

Examination of human albumin in the rat's serum after cell transplantation indicated persistent secretion of human albumin in the animals receiving hiPSC-EB-HLCs with or without inhibitors. In particular, at 72 hours after transplantation both groups of rats that received the clusters with or without inhibitors displayed human albumin in their serum at a concentration of 1.63±0.43 ng/mL and 0.20±0.05 ng/mL respectively. At 14 days after cell transplantation, the concentration of human albumin in the rats' serum increased in both experimental groups with and without inhibitors reaching the values of 28.20±7.8 ng/mL and 18.80±5.4 ng/mL respectively. The overall results for both experimental groups showed that human albumin was detected in nearly 80% of the survived animals receiving the hiPSC-EB-HLCs treated with WIF-1 and DKK-1 and 66% in the rats transplanted with the hiPSC-EB-HLCs differentiated without the two inhibitors. None of the control groups at any time showed human albumin in their serum (Table 1).

FIG. 6C and FIG. 6D show representative patterns of positive staining of human albumin in the livers of the hiPSC-EB-HLC transplantation group at 14 days post-transplantation. Spleen sections in all animals in this group were negative for human albumin staining. Co-expressions of all three human hepatic proteins (HNF-3 β, human albumin, and C-MET) by the transplanted hiPSC-EB-HLCs in these rat livers were seen throughout the examination period of 14 days post-transplantation using the immunohistochemical staining of the whole liver (FIG. 6E). The staining specificity was confirmed using human liver as a positive control (FIG. 6F).

Improved understanding of the events and the stage-specific inducing factors that are implicated in physiological hepatogenesis has contributed to the development of differentiation culture protocols to derive HLCs from hiPSCs in vitro. In general, existing protocols to differentiate hESCs or hiPSCs into HLCs are limited by two issues: low differentiation efficiency and high heterogeneity of the resultant cell populations. In one study (Agarwal, S. et al., Stem Cells 26, 1117-1127 (2008)), hESCs plated on adherence culture on MEF feeder layers underwent 2-step differentiation, first into definitive endoderm, then to hepatocytes on a collagen I matrix in serum-free medium through stepwise addition of inducing factors that were involved in early and late hepatic development. The differentiating cultures exhibited sequential expression of stage-specific hepatic genes, a hepatic differentiation yield of nearly 70%, in vitro functional hepatocyte characteristics, and repopulation of the remnant liver in a mouse model of liver injury. During the differentiation process, the differentiated cells demonstrated progressive loss of expression of the pluripotent markers Oct4 while gaining strong expression of early-stages hepatic proteins Sox17, FoxA2, and Gata4, followed by late-stage hepatic proteins albumin, CD26, and AAT, consistent with increased specification toward hepatic lineage. Despite these findings, the in vitro adherence culture-based hESC-derived HLCs retained the expression of immature markers for fetal hepatocytes and exhibited some functional deficiency (e.g., low P450 activities), suggesting incomplete differentiation or cell maturation under the described conditions. Similar to this study, there are other reports on the limitations of existing protocols (Song, Z. et al. Cell Res 19, 1233-1242 (2009); Sullivan, G. J. et al. Hepatology 51, 329-335 (2010); Takata, A. et al., Hepatol Int 5, 890-898 (2011); Touboul, T. et al., Hepatology 51, 1754-1765 (2010)). To date, differentiation of hiPSCs to cells equivalent to primary hepatocytes has not been achieved.

Conventional hepatic differentiation based upon 2D adherence culture has generated cell populations that differ from primary hepatocytes (Song, Z. et al. Cell Res 19, 1233-1242 (2009); Sullivan, G. J. et al. Hepatology 51, 329-335 (2010); Takata, A. et al., Hepatol Int 5, 890-898 (2011); Touboul, T. et al., Hepatology 51, 1754-1765 (2010)). 2D differentiation on planar substrates fails to capture the intricate structure of the 3D extracellular environment in native tissue, and therefore constrains the ability to generate cells of phenotypes and properties that closely mimic primary cells in vivo. During liver organogenesis, the liver bud is a 3D structure with dynamic cell-cell interactions among multiple cell types during development. Cell-cell interactions, particularly through E-cadherin positively impact hepatocyte maturation. Previous studies have shown that primary hepatocytes and hiPSC-derived HLCs grown in 3D culture retain their hepatic features better when compared to their counterparts in 2D culture (Vosough, M. et al. Stem Cells Dev 22, 2693-2705 (2013); Glicklis, R. et. al., Biotechnol Bioeng 67, 344-353 (2000); Ramaiahgari, S. C. et al., Arch Toxicol 88, 1083-1095 (2014); Sivertsson, L. et al., Stem Cells Dev 22, 581-594 (2013). Most of the published protocols for the hepatic differentiation of hiPSCs or hESCs into HLCs have paired 2D culture during early stage of differentiation with subsequent 3D culture to promote assembly of differentiated cells for final maturation. The differentiation protocol disclosed herein was performed completely in 3D culture, using a new ROCKi-free and spin-free technique for EB formation. When compared to 2D adherence culture-based differentiation, 3D culture-based differentiation using hiPSC-EBs offers several advantages including greater capacity for high cell density by obviating the cell-cell contact inhibition and growth surface area restrictions in 2D, and promoting maturation of HLCs by cell-cell contact. In addition, differentiated cells in the form of clusters do not require enzymatic or mechanical dissociation before use, thus reducing potential cell damage/loss due to further processing. Clusters of differentiated cells generated in 3D culture are clearly visible, easy to transport, and readily injectable. Our differentiated hiPSC-EB-HLCs in the form of clusters did not demonstrate any core necrosis up to 1,000 μm in diameter, suggesting that the permeability level of the clusters was sufficient to allow oxygen/nutrient exchange and diffusion.

Despite the sequential administration of inducing growth factors involved in physiological hepatogenesis to drive the differentiation of hiPSCs through different stages, none of the previously published hepatic differentiation protocols address the inhibition of the Wnt pathway that occurs during in vivo liver organogenesis. The effect of Wnt/β-catenin signaling on cell specification toward specific lineages, including hepatocytes, is widely seen during embryogenesis across species. During early liver development, β-catenin expression is highest at E10-E12, followed by a reduction after E16. In hepatogenesis, Wnt modulation occurs at a late stage of cell differentiation, and in conjunction with β-catenin, is crucial in dictating the differentiation of liver progenitor cells (i.e., hepatoblasts) toward hepatocytes or cholangiocytes. When activated, the Wnt/β-catenin pathway drives hepatoblasts toward cholangiocytes, while when inhibited, it drives hepatoblasts toward hepatocytes. These effects of the Wnt/β-catenin pathway have allowed manipulation at the fate-determining hepato-biliary stage during differentiation to increase the yield in one or the other phenotype. By incorporating the inhibitors of the Wnt/β-catenin pathway into the differentiation protocol, it is possible to offset the balance of fate specification into hepatocytes vs. cholangiocytes, therefore enhancing hepatocyte production. The Wnt/β-catenin pathway is regulated by two classes of antagonists. One is the secreted frizzled-related protein (sFRP) family (e.g., WIF-1) which blocks Wnt signaling through binding to Wnt proteins, and the other is the Dickkopf (DKK) class (e.g., DKK-1) which blocks Wnt signaling through inhibiting the formation of the Wnt-induced Frizzled-LPR5/6 complex. Wnt proteins are also grouped into two classes: canonical and noncanonical, based upon their activity in cell lines and in vivo assays. In theory, sFRP family inhibits both canonical and noncanonical pathways, whereas DKK class specifically inhibits the canonical pathway. In particular, DKK-1 inhibits Wnt-induced stabilization of β-catenin, and may be specific to the Wnt/β-catenin pathway. As disclosed herein, inhibitors from both classes were administered, i.e., WIF-1 and DKK-1, in the hope that they may act synergistically in blocking the Wnt/β-catenin pathway.

The stage-specific temporal gene and protein expression profiles of our hiPSC-EB-HLCs are consistent with previous reports, confirming a stepwise differentiation into mature hepatocytes using our protocol. The protocol disclosed herein recapitulates in vitro the four stages seen in liver development during normal embryogenesis, starting with the pluripotent state (PS), definitive endoderm (DE), foregut endoderm (FE), hepatic progenitors or hepatoblast (HP), and mature hepatocytes (MH). There was overlap in the gene expression of each stage. Addition of the two inhibitors of the Wnt/β-catenin pathway at a late stage during the hEB-based 3D hepatic differentiation program has increased the commitment of hepatoblasts toward mature hepatocytes while suppressing the production of other cell types, specifically cholangiocytes. There was a significantly higher yield of mature hepatocytes (over 80%) following differentiation in the presence of both Wnt inhibitors (WIF-1 and DKK-1) relative to the differentiated cells in the absence of both Wnt inhibitors highlighted by the presence of human albumin production. The Wnt inhibitors also address the issues of incomplete differentiation and maturation that are associated with conventional protocols. In vitro, the hiPSC-EB-HLCs displayed a full spectrum of functionality of mature hepatocytes including albumin secretion, detoxification and metabolism through the P450 enzyme family, AFP secretion, Fibrinogen secretion, and lipid and glycogen storage for both groups with and without WIF-1 and DKK-1. Among them are key functions of mature hepatocytes, such as LDL uptake indicative of fatty acid absorption for lipogenesis, glycogen uptake and storage, triglyceride storage as an energy reservoir, and ICG uptake and subsequent clearance showing the ability to metabolize certain substances. ICG is an organic anionic dye that is exclusively eliminated by the liver. One of the most important functions of hepatocytes is detoxification and metabolism through the P450 enzyme family. This function is essential in vivo and in vitro for pharmaceutical screening as it helps to determine drug toxicity and tolerance. In addition to constitutive activity, the hiPSC-EB-HLCs from both experimental groups demonstrated the ability to up-regulate specific P450 family CYP enzyme isoforms in response to specific inducers. In the studies disclosed by the examples herein, three common and physiologically important P450 isoforms were tested. These findings strongly suggest that the cells have undergone maturation to a mature hepatocyte phenotype and are functional in terms of detoxification and metabolism and response to major external stimuli.

The hiPSC-EB-HLCs that were generated in the 3D culture disclosed herein was performed in a scalable manner capable of rescuing animals from acute liver failure in a rat model. Liver failure causes a physiological severe deficiency in hepatic function, and is associated with significant mortality and morbidity worldwide. The only effective treatment to date is liver cellular and solid organ transplantation. Shortage of liver donors and a low efficiency of primary hepatocytes cell transplantation therapy represent insurmountable obstacles for treatment. In the examples disclosed herein, at 2 weeks post-transplantation, no hiPSC-EB-HLCs (human albumin-positive cells) were seen in the spleen, the original site of injection, yet numerous hiPSC-EB-HLCs were clearly seen in the recipient rat livers. These findings are in line with previous reports of intrasplenically transplanted primary hepatocytes of human or animal origin leaving the spleen for nidation in the liver chords, suggesting the replication of a key feature of primary hepatocytes by the hiPSC-EB-HLCs. The transplanted hiPSC-EB-HLCs persistently secreted human albumin into the host plasma throughout the examination period (72 hours and 14 days), and successfully bridged the animals subjected to acute liver failure through the critical period for survival, providing a promising clue of integration and full in vivo functionality of these cells. In particular, the animals transplanted with hiPSC-EB-HLCs treated with the two inhibitors displayed a higher concentration of human albumin in their serum compared with the ones that were transplanted with hiPSC-EB-HLCs without inhibitors.

The experiments described above were carried out comparing the hiPSC-EB-HLCs differentiated using two protocols with and without WIF-1 and DKK-1. The comparison between the two conditions showed that when WIF-1 and DKK-1 were added, the differentiation process was enhanced as demonstrated by improved hepatic functionality of the resultant hiPSC-EB-HLCs.

Taken together, the stepwise 3D spheroid culture-based hepatic differentiation protocol as disclosed herein, involving two inhibitors of the Wnt/β-catenin pathway at a late stage during differentiation, has resulted in hiPSC-EB-HLCs that not only bear the genetic and proteomic signatures of adult primary human hepatocytes, but also mature hepatocyte-like functionality both in vitro and in vivo. The differentiation program is readily scalable and highly efficient. The resultant cell population is homogeneous, fully differentiated, and matured. These cells likely provide viable substitutes for primary human hepatocytes in regenerative medicine and pathophysiological studies, as well as pharmacological screening and drug discovery.

### Example 4: Embryoid Body Formation

The invention provides methods for reproducibly generating large numbers of mature hepatocytes that are suitable for transplant. This example provides a detailed description of such methods. Human induced pluripotent stem cells (hiPSCs) are a foreskin fibroblast-derived cell line iPS(foreskin)-3 purchased from WiCell Research Institute (Madison, Wis. —cat # WB0002) and cultured in a chemically defined stem cell medium, TeSR2 basal medium with TeSR2 supplements (Stem Cell Technologies, Ontario, Canada) on Vitronectin coated plates (Stem Cell Technologies, Ontario, Canada).

For the formation of the human embryoid bodies, a special agarose mold was used. The Aether™ agarose-mold was created by using PDMS micro-molds where 0.5 mL of a 2% molten agarose solution (Sigma-Aldrich, Cat #: A2929) was pipetted into the micro-molds which were filled completely. The agarose was allowed to gel for about 2 minutes and then placed into a 24 multi-well plate in a sterile environment. Aether agarose-molds possess a specific round bottomed convexity that allows the formation of perfectly spherical embryoid bodies which are created starting from a specific cell seeding density concentration of 1.2×10^{6/mold}/3.5×10^{4/well} single cell suspension.

The seeded single cell suspension of hiPSCs into the Aether™ agarose-mold were incubated for a period of time that went from 12 hours, up to 24 hours (not exceeding 24 hours). In contrast to other techniques that are commonly used, the protocol disclosed herein does not use a Y-27632 RHO/ROCK pathway inhibitor, and does not use any centrifugation force to allow the aggregation of the hiPSCs single cell suspension. The methods of the invention allow human embryoid bodies to be obtained that are free from any adverse effect that ROCKi or centrifugation could pose for their future use in human cell therapies. At the end of the incubation time, the formed hEBs are extracted from the Aether™ agarose-mold and put in suspension culture using a specific Aether™ chemically defined and serum-free formulated medium composed of Iscove's Modified Dulbecco's Media (IMDM) supplemented with F-12 Nutrient Mixture (Ham), 100 U/ml⁻¹ penicillin, and 0.1 mg ml⁻¹ streptomycin (Gibco, Cat #15140122) and 55 μM 1-Thioglycerol supplemented with 100 μg/ml of Oleoyl-L-α-lysophosphatidic acid sodium salt (LPA) (Sigma-Aldrich, Cat # L7260-5MG), 1 gr/L recombinant human insulin (Sigma-Aldrich, Cat #91077C-100MG), 0.55 gr/L recombinant human transferrin (Sigma-Aldrich, Cat # T3705-1G), 0.00067 g/L sodium selenite (Sigma-Aldrich, Cat # S5261-100G), and 11 gr/L sodium pyruvate (Sigma-Aldrich, Cat # S8636-100ML). Such chemically defined and serum-free medium allows the cells to create the human embryoid bodies in a xeno-free condition.

### Example 5: Human Embryoid Body Formation: hiPSCs Interlaced with Human Adipose-Tissue-Derived Endothelial Cells (hATECs)

In another embodiment, the invention provides human embryoid bodies containing hiPSCs and hATECs. This approach ameliorates the maturation/differentiation in vitro and post-transplantation nidation in vivo of the differentiated human embryoid bodies. The formation of human embryoid bodies was performed using 8.16×10^{5/mold}/2.3×10^{4/well} of hiPSCs single cell suspension interlaced with 4.08×10^{5/mold}/1.16×10^{4/well} of hATECs single cell suspension which results in a specific ratio between hiPSC and hATEC of 1:3. Such ratio allows the maximum combinatorial effect to be obtained during the differentiation process of hiPSCs and hATECs. This variation of human embryoid body formation was also carried out using the Aether™ chemically defined and serum-free medium described in Example 4. In this case, the hiPSCs and hATECs were incubated for 24 hours to facilitate human embryoid body formation.

### Example 6: Human Embryoid Bodies with hiPSC+ATECs Coated with Human Mesenchymal Stem Cells (hMSCs)

The invention further provides methods for obtaining human embryoid bodies containing hiPSCs with or without hATECs, and coated with hMSCs. To protect the differentiated human embryoid bodies, (with or without hATECs), from the attack of the host immune system after transplantation, at completion of the differentiation protocol described in Example 7, the differentiated human embryoid bodies were coated with a thin layer of degradable and bio-compatible hydrogel that degrades over time, preventing the invasion of hMSCs within the differentiated human embryoid bodies. The hydrogel structure was prepared as follows: (1) P-nitrophenyl carbonated dextran (Dex-PNC) and thiolated dextran (Dex-SH) was synthesized. (2) A disulfide bond containing an aminated dextran Dex-SS-NH2 was then prepared via thiol-disulfide exchange reaction of dextran-SH and S-(2-pyridylthio) cysteamine (PDA) hydrochloride. (3) The redox-responsive amphipathic dextran (Dex-SSDCA) was then synthesized by the condensation reaction between the carboxyl of deoxycholic acid (DCA) and the amine of Dex-SS-NH2. The degradation of the hydrogel may subsequently be finely tuned from 3 days to 2 weeks by controlling the molecular weight and degree of substitution of DCA. The human embryoid bodies containing hiPSCs with or without hATECs are then mixed with 2 mL of Dex-SSDCA solution in DMSO at a concentration of 10 mg/mL. The mixture will be stirred overnight.

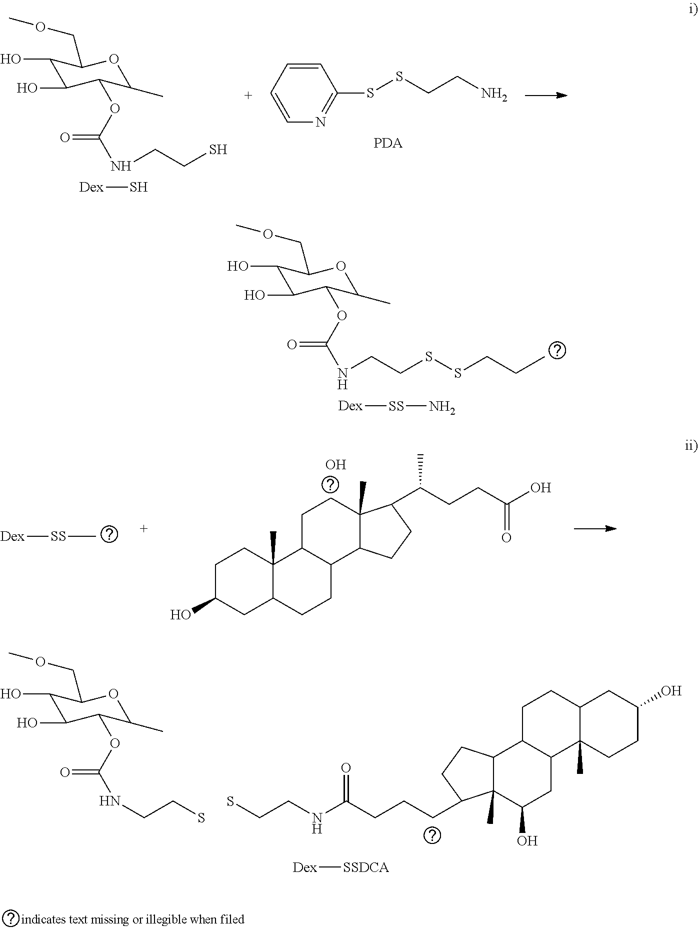

Synthesis of Dex-SSDCA

The hydrogel, containing a coated layer of differentiated human embryoid bodies, was then transferred into the Aether™ agarose-molds and co-cultured with 4.4×10^{5/mold} of hMSCs for 24 hours in order to create a protective hMSCs coating outer layer. Since hMSCs are anchor-dependent cells, these cells have to attach on a surface in order to survive. The only attachable surfaces available to the hMSCs were the surfaces of the differentiated human embryoid bodies. Using this simple method, a single cell thick layer (about 26 μm) of hMSCs was coated on the differentiated human embryoid bodies. To ensure the hMSCs are kept in place as a surrounding capsule for the spheroids, a second degradable and biocompatible hydrogel layer was added on the outer layer of the hMSCs coated islet-like clusters. The second hydrogel layer prevents migration of the human embryoid bodies away from the hMSCs coated islet-like clusters.

### Example 7: Differentiation of Human Embryoid Bodies Derived from hiPSC Interlaced with Human Adipose-Tissue Endothelial Cells (hATECs) into Hepatocyte Like Clusters

The four-stage in vitro hepatic differentiation protocol sought to recapitulate the changes that occur during embryogenesis. The four stages of hepatic differentiation are definitive endoderm, foregut endoderm, hepatobiliary progenitor and committed hepatocyte. Each stage of the differentiation protocol last four days with two every-other-day medium changes and addition of the soluble differentiation factors. The details are provided herein below. The basal differentiation medium used in culture was a specific Aether™ chemically defined and serum-free formulated medium composed of IMDM with F-12 Nutrient Mixture (Ham), 100 U/ml⁻¹ penicillin, and 0.1 mg ml⁻¹ streptomycin (Gibco, Cat #15140122) and 55 μM 1-Thioglycerol supplemented with 100 μg/ml of Oleoyl-L-α-lysophosphatidic acid sodium salt (LPA) (Sigma-Aldrich, Cat # L7260-5MG), 1 gr/L recombinant human insulin (Sigma-Aldrich, Cat #91077C-100MG), 0.55 gr/L recombinant human transferrin (Sigma-Aldrich, Cat # T3705-1G), 0.00067 g/L sodium selenite (Sigma-Aldrich, Cat # S5261-100G), and 11 gr/L sodium pyruvate (Sigma-Aldrich, Cat # S8636-100ML).

Differentiation towards definitive endodermal stage was promoted through the inhibition of the sonic hedgehog (Shh) pathway. When active, the Shh pathway promotes the differentiation of foregut cells, while when inhibited the Shh pathway drives cells toward a definitive endodermal phenotype. Activin A is a soluble factor belonging to the TGF-β superfamily and, like the other members of this superfamily, interacts with two types of transmembrane receptors on the cells surface (types I and II) that possess intrinsic serine/threonine kinase activity in their cytoplasmic domains. Activin A binds to type II receptor and begins a cascade reaction that leads to the recruitment, phosphorylation and activation of the type I receptor. Activated type I receptor then interacts with the type II receptor and together, phosphorylate Smad2 and Smad3. Smad3 then moves into the nucleus, where it interacts with Smad4 through multimerization, resulting in their modulation as a complex of transcription factors responsible for the expression of a large variety of genes. Studies on the action of this complex of transcription factors have shown that activin A, at a given concentration, allows the inhibition of the Shh pathway. Hence, through the action of activin A, it is possible to obtain the differentiation of the hiPSCs towards a definitive endodermic phenotype. The concentration at which activin A has been shown to induce differentiation toward definitive endoderm is 100 ng/ml for a period of four days. At low concentrations (50 ng/ml), activin A appears to possess pluripotential maintenance activity similar to bFGF, another component of TGF-β superfamily. To enhance the activity of activin A in promoting definitive endoderm formation, two soluble factors belonging to the same superfamily were added, TGF-β 1 and bFGF. These factors were added immediately embryoid body formation and for a period of four days. (See Hepatic Differentiation Procedure, below) The synergistic action of these three factors led to greater definitive endoderm formation. The doses at which these three factors were used are the following: 100 ng ml⁻¹ Activin-A, 10 ng ml⁻¹ basic FGF and 10 ng ml⁻¹ TGF-β (all from PeproTech, Rocky Hill, N.J.).

The next step in liver differentiation that leads toward an endocrine phenotype is managed by the Notch pathway, which when active represses the differentiation of liver progenitors cells, by keeping them in a “stand-by” state, while if blocked will lead to the formation of endocrine liver cells. Neurogenin 3 (Ngn3) belongs to the basic helix-loop-helix (bHLH) transcription factors family that is involved in the development of endocrine cells. It has been shown that transgenic mice that overexpress Ngn3 in early phases of their development show a marked increase in the formation of endocrine cells, indicating that Ngn3 induces the differentiation of liver cells precursors. Ngn3 activity appears to be also involved in the Notch pathway inhibition. Two factors that play an important role in activating Ngn3 are BMP4 and FGF4. By adding these two factors at the second stage of this protocol, the expression of Ngn3 has been upregulated, therefore modulating and blocking the Notch pathway. The specific doses at which BMP4 and FGF4 were added are 10 ng ml⁻¹ FGF-4 (PeproTech) and 10 ng ml⁻¹ BMP-4 (Invitrogen). These two factors, BMP4 and FGF4, were added four days after the initiation of the treatment with Activin A and for a period of four days. (See Hepatic Differentiation Procedure, below)

Following endodermal commitment, absence of Wnt signaling is essential for the commitment of the liver progenitor cells (hepatoblasts) into hepatocyte cells. Wnt and β-catenin demonstrate individual as well as the combined effects in controlling postnatal liver development. Increased β-catenin translocation to the nucleus correlates with an increase in cell proliferation, whereas the Wnt pathway is considered as the major regulator of polarity and cell fate specifications. The effect of Wnt and β-catenin on liver embryogenesis follows a highly temporally regulated profile. When combined, the Wnt/β-catenin pathway plays an important role in the hepato-biliary differentiation toward hepatocytes, whereas stabilization of β-catenin alone leads to increased propensity toward cholangiocytes over hepatocytes. Through Wnt/β-catenin inhibition, it is possible to promote progression to hepatocytes at the hepato-biliary differentiation stage. During phase II of liver development, hepatoblasts or hepatic progenitors undergo expansion while maintaining their de-differentiated state. Commitment to a hepatic fate is regulated by an array of the liver-enriched transcriptional factors that are present during phase III. Current conventional differentiation protocols follow a stepwise process from the initial endoderm formation, passing through hepatic progenitor cell induction, toward a mature hepatic phenotype without taking into account the important role of Wnt/β-catenin inhibition. The effect of Wnt/β-catenin signaling on cell specification toward specific lineages, including hepatocytes, is widely seen during embryogenesis across species. During early liver development, β-catenin expression is highest at E10-E12, followed by a reduction after E16. In hepatogenesis, Wnt modulation occurs at a late stage of cell differentiation, and in conjunction with β-catenin, is crucial in dictating the differentiation of liver progenitor cells (i.e., hepatoblasts) toward hepatocytes or cholangiocytes. When activated, the Wnt/β-catenin pathway drives hepatoblasts toward cholangiocytes, while when inhibited, it drives hepatoblasts toward hepatocytes. These effects of the Wnt/β-catenin pathway have allowed manipulation at the fate-determining hepato-biliary stage during differentiation to increase the yield in one or the other phenotype. By incorporating the inhibitors of the Wnt/β-catenin pathway into the differentiation protocol, it is possible to offset the balance of fate specification into hepatocytes vs. cholangiocytes, therefore enhancing hepatocyte production. The Wnt/β-catenin pathway is regulated by two classes of antagonists. One is the secreted frizzled-related protein (sFRP) family (e.g., WIF-1) which blocks Wnt signaling through binding to Wnt proteins, and the other is the Dickkopf (DKK) class (e.g., DKK-1) which blocks Wnt signaling through inhibiting the formation of the Wnt-induced Frizzled-LPR5/6 complex. Wnt proteins are also grouped into two classes: canonical and non-canonical, based upon their activity in cell lines and in vivo assays. In theory, sFRP family inhibits both canonical and non-canonical pathways, whereas DKK class specifically inhibits the canonical pathway. In particular, DKK-1 inhibits Wnt-induced stabilization of β-catenin, and may be specific to the Wnt/β-catenin pathway. In the current protocol, inhibitors were administrated from both classes, i.e., WIF-1 and DKK-1, as they may act synergistically in blocking the Wnt/β-catenin pathway.

The specific doses at which the two Wnt pathway inhibitors are effective are 1 μg ml⁻¹ of WIF-1 (R&D System, Minneapolis, Minn.) and 0.1 μg ml⁻¹ of DKK-1 (PeproTech), which serve to suppress the Wnt signaling and promote the differentiation of hepatoblasts into hepatocyte-like cells in the third stage of our differentiation protocol. These two factors, WIF-1 and DKK-1, were administered immediately after the administration of BMP4 and FGF4, and for a period of four days. (See Hepatic Differentiation Procedure, below)

For the final maturation of the hepatocytes-like cells, the presence of HGF and Oncostatin M determines the terminal differentiation into mature hepatocytes. Oncostatin M induces maturation of fetal hepatic cells derived from the embryonic day 14.5 (E14.5) liver in vitro. Hepatic maturation induced by Oncostatin M is mediated through STAT3, since expression of hepatic differentiation markers is efficiently inhibited by expression of a STAT3 Inhibitor in fetal hepatic culture. For example, STAT3 Inhibitors include SH2 domain inhibitors or dimerization inhibitors (SDIs, site B), DNA binding domain inhibitors (DBDIs, site C), N-terminal domain inhibitors (NDIs, site D), and the indirect targeting of the upstream components of the STAT3 pathway (site A, tyrosine phosphorylation inhibitors, TPIs). Hepatocyte growth factor (HGF) was shown to directly stimulate proliferation of adult hepatocytes in vitro. However, HGF did not activate STAT3 in fetal hepatic cells and expression of STAT3 failed to inhibit expression of the liver differentiation marker gene induced by HGF. Although both OSM and HGF induced hepatic differentiation, their signaling mechanisms are quite different. The signal molecules activated by HGF complete the functions of OSM in liver development; therefore the synergistic effect of Oncostatin M with HGF is needed to lead the maturation of hepatocyte-like cells in the differentiation protocol. The specific doses at which Oncostatin M and HGF were effective are 50 ng ml⁻¹ for HGF (PeproTech) and 30 ng ml⁻¹ for Oncostatin M (PeproTech). The whole differentiation process was carried out in suspension culture. The differentiated hepatocyte-like clusters obtained were ready to be utilized for any type of application, both in vitro testing and clinical therapy.

### Example 8: WIF-1 and DKK-1 Drive hiPSC-EB Differentiation into Hepatocyte-Like Cells

The studies of this example show that this novel differentiation protocol using WIF-1 and DKK-1, coupled with a 3D suspension culture of hiPSC embryoid bodies in combination with hECs is able to drive hiPSC-EB differentiation into hepatocyte-like cells in a scalable manner. The differentiated hiPSC-EB-HLC of this example displayed-both in vitro and in vivo-most of the main physiological functions of mature human hepatocytes, making them suitable for in vitro studies as well as pharmaceutical drug testing and cell therapy.

Human embryoid bodies (hEBs) were derived using the previously developed ROCKi-free/Spin-free technique that allowed for scalable production and uniform hEBs in large quantities (Pettinato, G. Sci. Reports, 4:7402, Dec. 10, 2014). For the studies of this example, hiPSCs were interlaced with human endothelial cells (hECs) in the same hEBs. Both hiPSCs and hECs were visualized within the same hEBs using live dyes such as DiO (green) and DiI (red) 24 hours post hEBs formation.

The differentiation protocol was designed to recapitulate developmental stages of the liver during embryogenesis in vivo (Pettinato, G., et al. Sci Rep. 2016 Sep. 12; 6:32888). Two novel Wnt/Beta-catenin inhibitors, WIF-1 and DKK-1, were used to drive the hepatoblast to become mature hepatocyte-like cells.

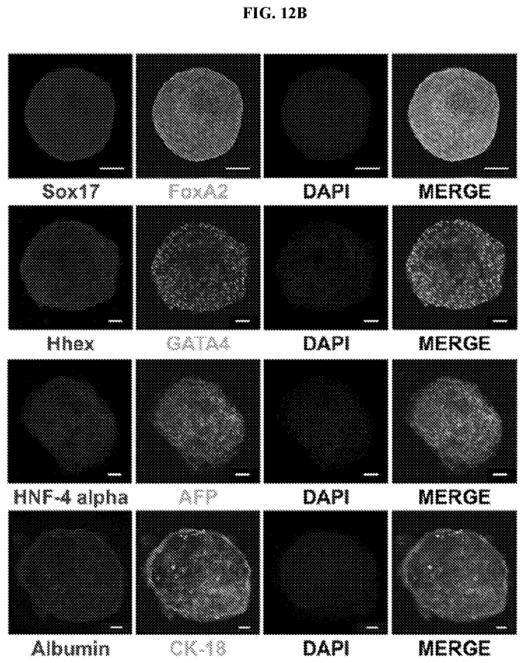

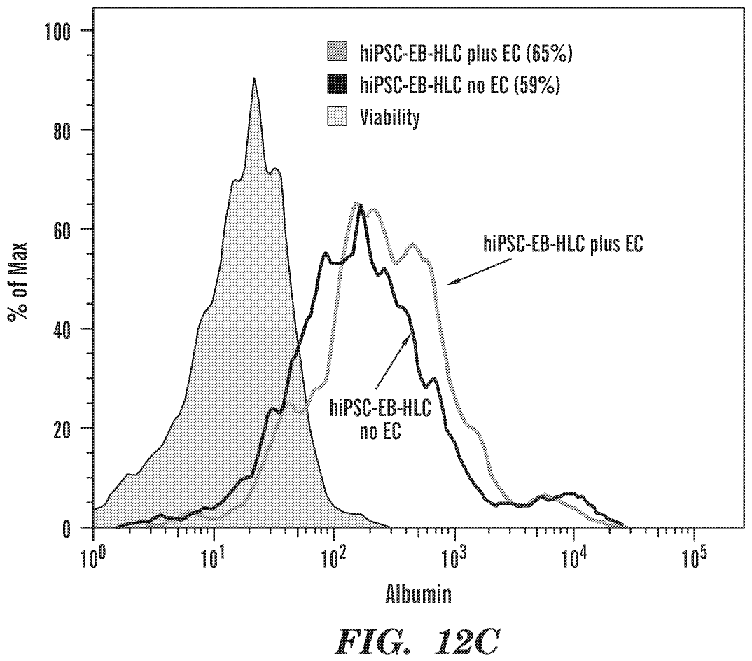

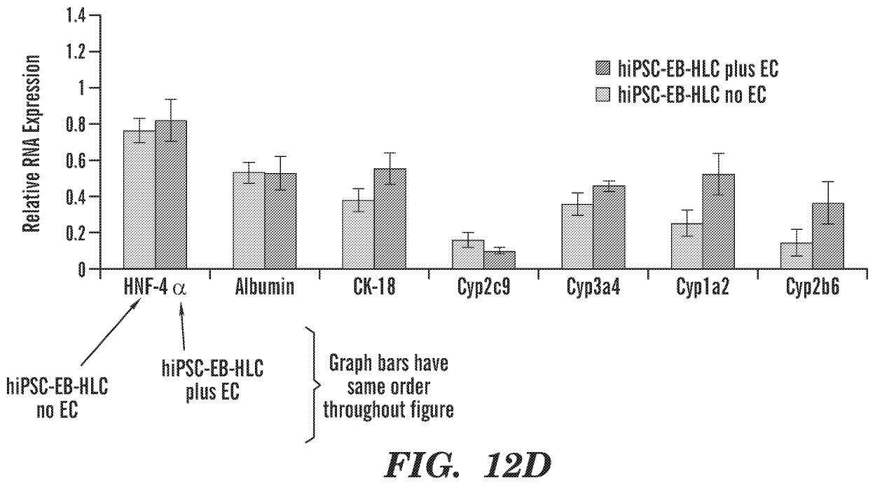

To confirm the stepwise differentiation of hiPSC-EB-HLCs interlaced with hECs into mature hepatocyte-like cells, immunofluorescence for various markers was used to demonstrate the differentiation of the hiPSC-EB-HLC plus hECs into mature hepatocyte-like cells (FIG. 12A, FIG. 12B, FIG. 12C). Staining was observed for SOX17 and FOXA2, which are markers for the endodermal stage; HHEX and GATA4 which are markers for the foregut endoderm; AFP and HNF-4a which are markers for hepatic progenitor cells; and ALBUMIN and CK-18 which are markers for mature hepatocyte-like cells. FIGS. 12A and 12B provide a comparison for the maturation steps between hEBs with hiPSCs only (A) and hEBs with hiPSCs interlaced with hECs (B) displaying the presence of stage specific markers. FIG. 12C provides a FACS analysis for albumin between the two experimental conditions with and without hECs. This analysis showed a higher percentage of albumin positive cells in the presence of hECs. FIG. 12D shows results of quantitative RT-PCR analysis which found greater expression of several genes when hECs were interlaced with hiPSCs.

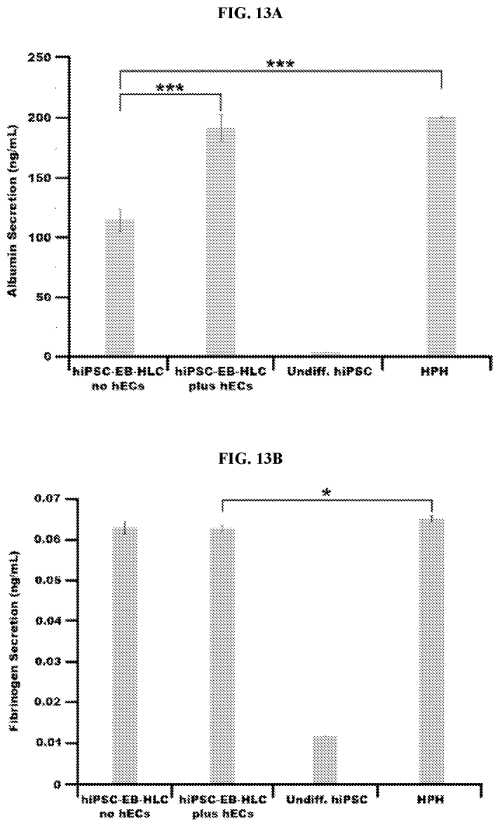

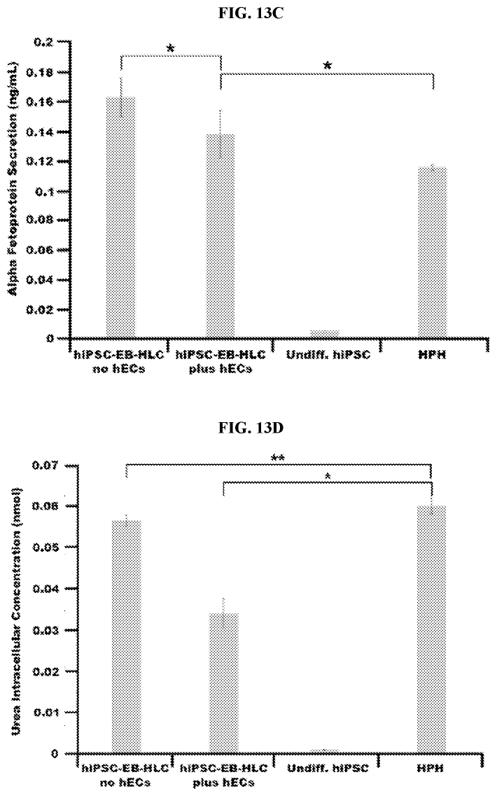

The cell media was assayed for the presence of albumin (FIG. 13A) fibrinogen (FIG. 13B) and alpha fetoprotein (AFP) (FIG. 13C) secreted into the medium by hiPSC-EB-HLC for both conditions (i.e., the two conditions refer to conditions with or conditions without inhibitors (WIF-1 and DKK-1); also, the experiments were carried out using hiPSC-EB-HLCs with and without hECs). The intracellular concentration of urea was also detected after differentiation (FIG. 13D). The results obtained from the analysis of all the markers showed similar levels compared to human primary hepatocytes (HPHs).

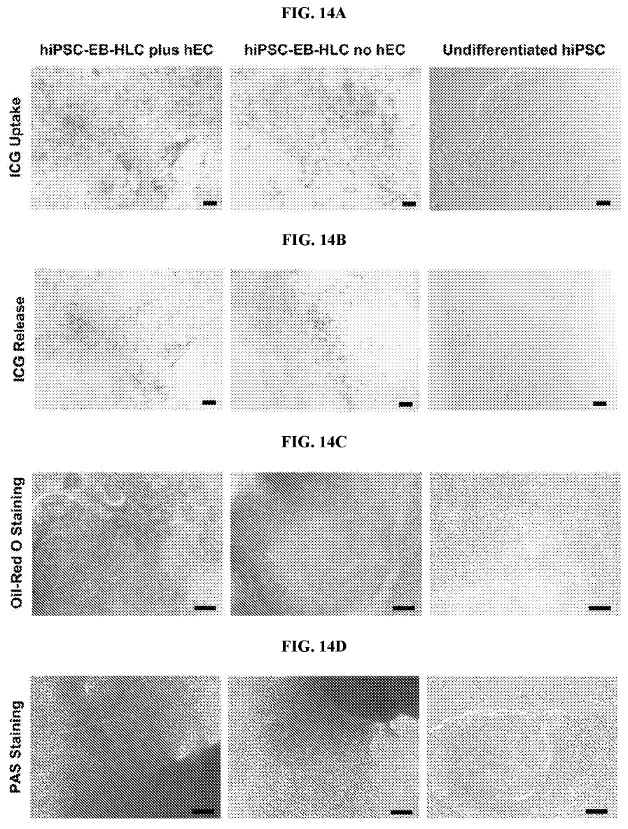

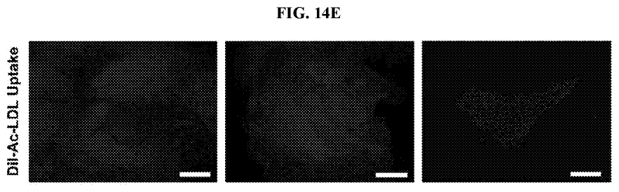

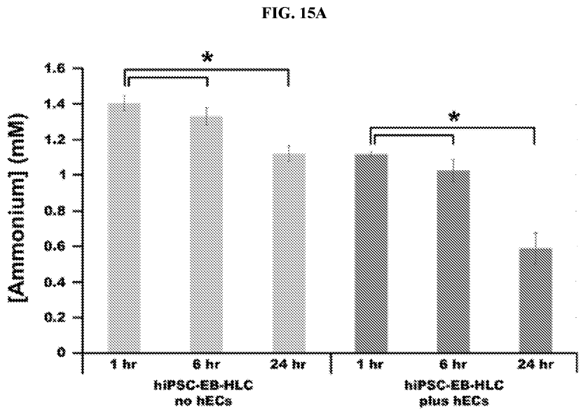

hiPSC-EB-HLCs with and without hECs were assayed for Indocyanine green (ICG—Cardiogreen) uptake (FIG. 14A); ICG release after 6 hours (FIG. 14B); Oil-Red O staining, which provided an assessment of the cytoplasmic accumulation of neutral triglycerides and lipids (FIG. 14C); glycogen storage was confirmed by PAS staining (FIG. 14D); and acetylated low-density lipoprotein (DiI-ac-LDL) uptake showed the presence of LDL vesicles in the differentiated cells (FIG. 14E) and did not displayed any positive staining for any of the conditions tested (right side). Scale bar 100 μm.

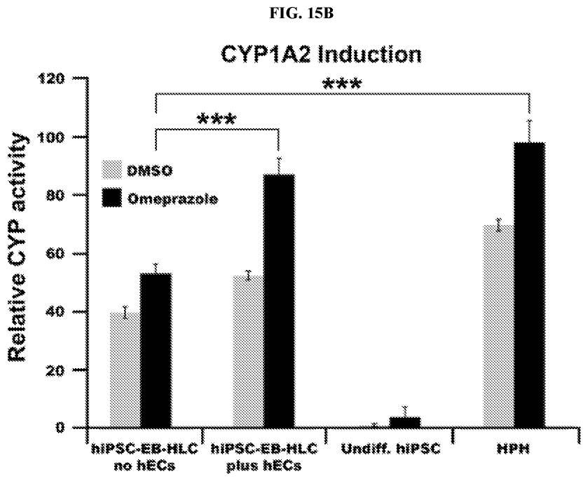

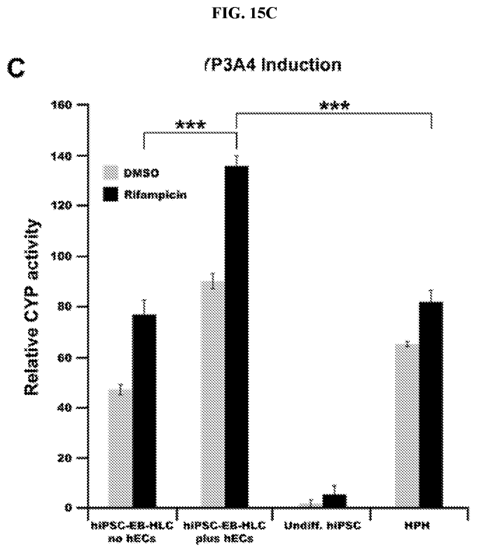

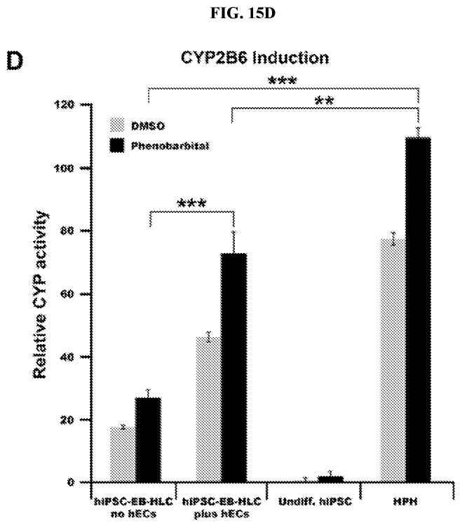

Ammonia metabolism via the urea cycle is an essential function of hepatocytes. Ammonia metabolism was evaluated by assaying changes in ammonium concentration in the cell culture supernatant for both experimental conditions over a 24-hour period after addition of ammonium chloride of known concentration. Several cytochromes P450 enzymes were evaluated by incubating the cells with different inducers: Omeprazole for CYP1A2, Rifampicin for CYP3A4 and Phenobarbital for CYP2B6 over a 72-hour period. DMSO was used as control to test the basal activity of the different CYP450. hEBs interlaced with hECs displayed a higher induction of all the Cytochromes P450 enzymes (FIGS. 15A-15D).

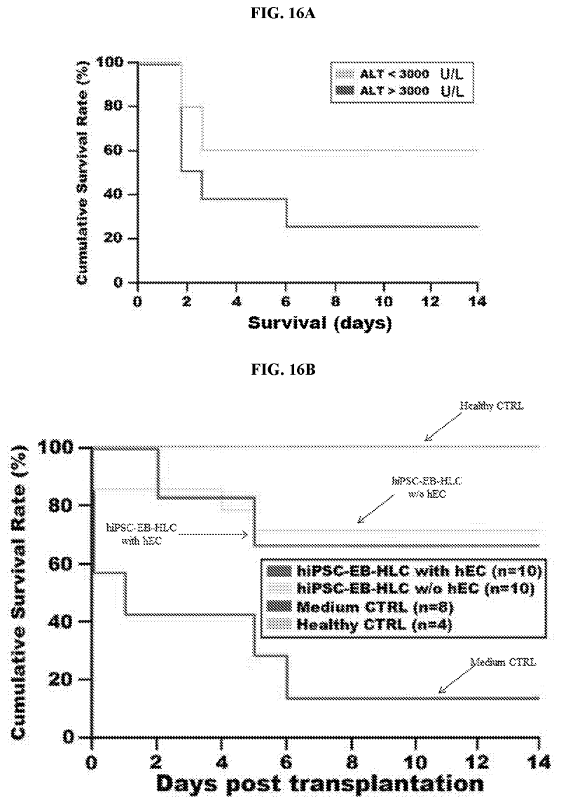

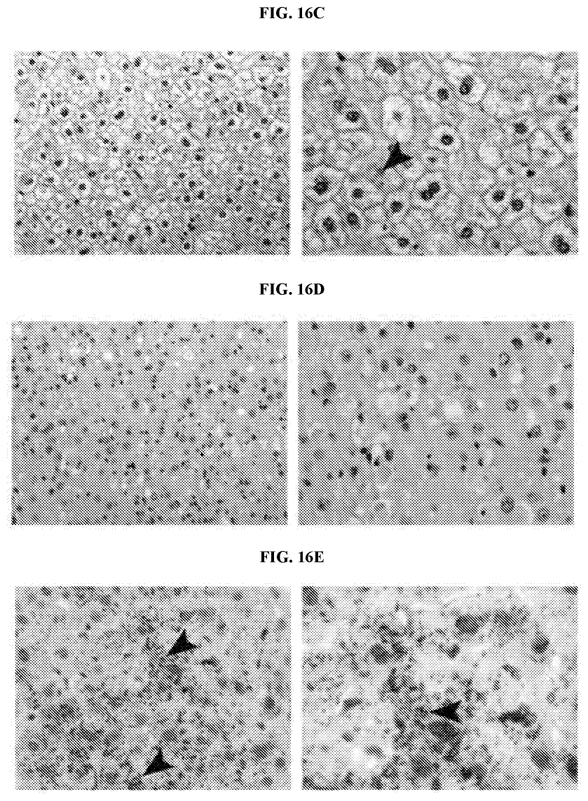

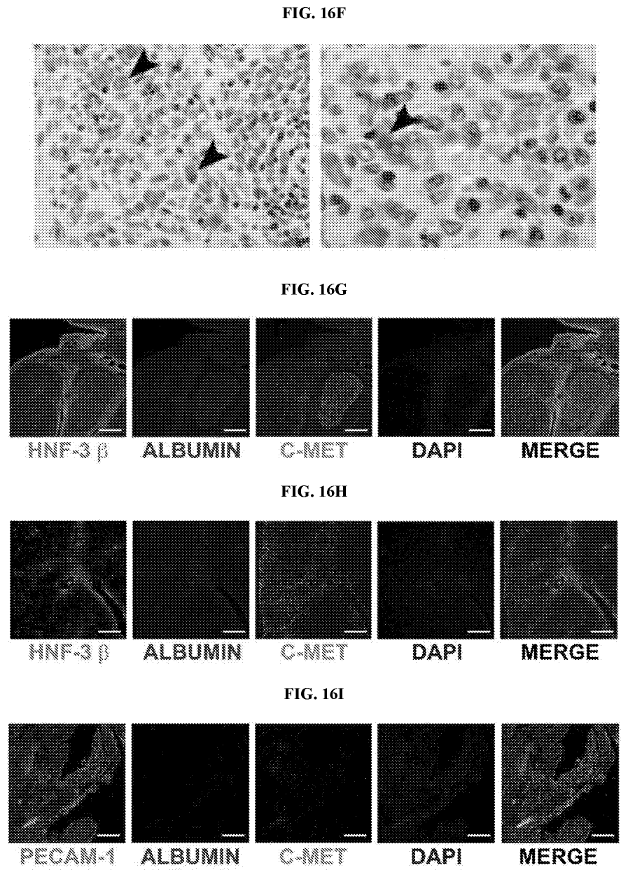

Next, the d-galactosamine-induced rat animal model of acute liver failure was used to determine the therapeutic effects of hiPSC-EB-HLC of both conditions with and without hECs. Alanine aminotransferase (ALT) was used as a marker of liver injury. FIG. 16A shows a Kaplan-Meier survival curve for model assessment without transplantation. Eight out of nine animals that had incurred liver injury with an ALT level>3,000 U/L 1 day post-d-galactosamine injection had a 3-day mortality, compared with two out of five animals in those with ALT<3,000 U/L. Animals that received hiPSC-EB-HLC with hECs transplantation had greater survival compared with the group without hECs (P<0.05) (FIG. 16B). Using immunohistochemical staining, hiPSC-EB-HLC with hECs in liver and spleen sections could be detected in animals sacrificed post-transplantation (FIG. 16C, FIG. 16D, FIG. 16E, FIG. 16F, FIG. 16G, FIG. 16H, FIG. 16I). These experiments confirm the therapeutic effects of transplantation of hiPSC-EB-HLC with hECs to increase survival in a liver injury setting.

### Example 9: HLCs and Coagulation Factor Secretion

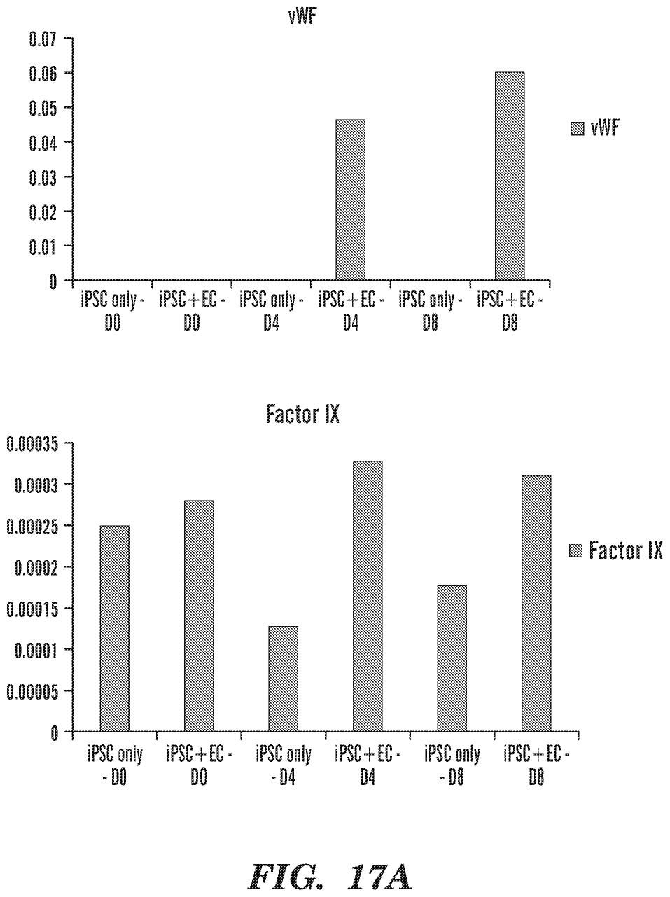

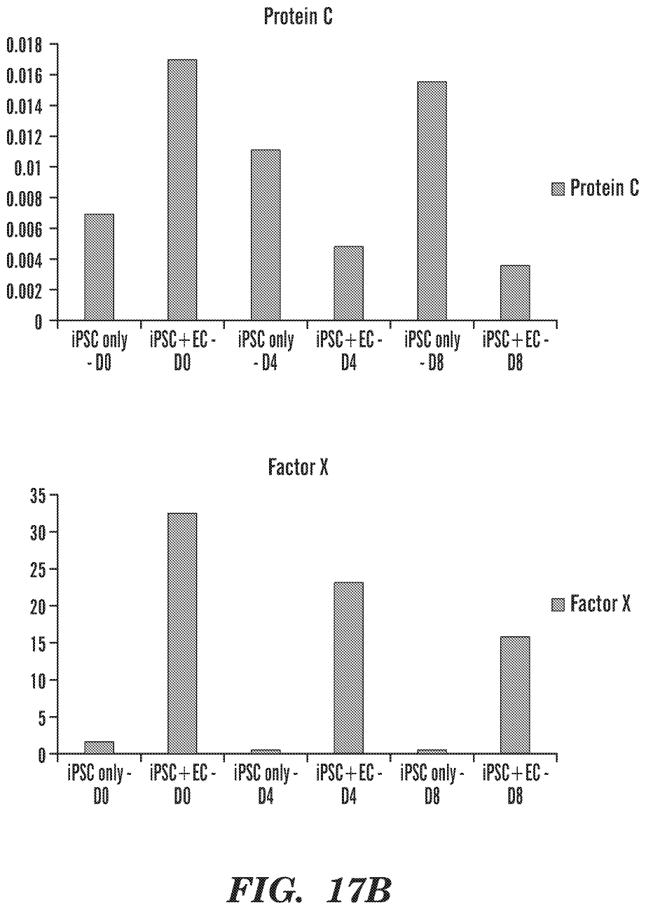

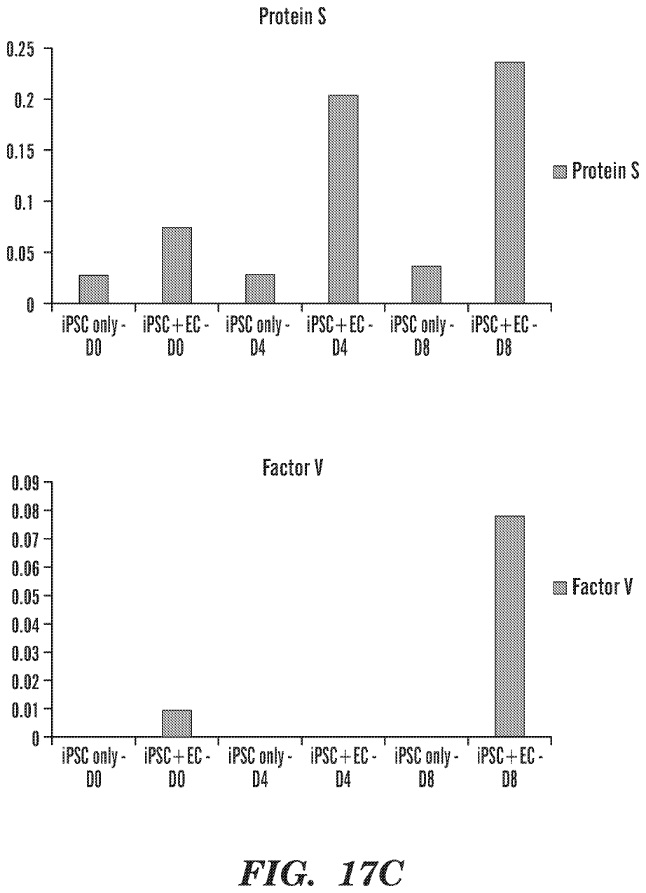

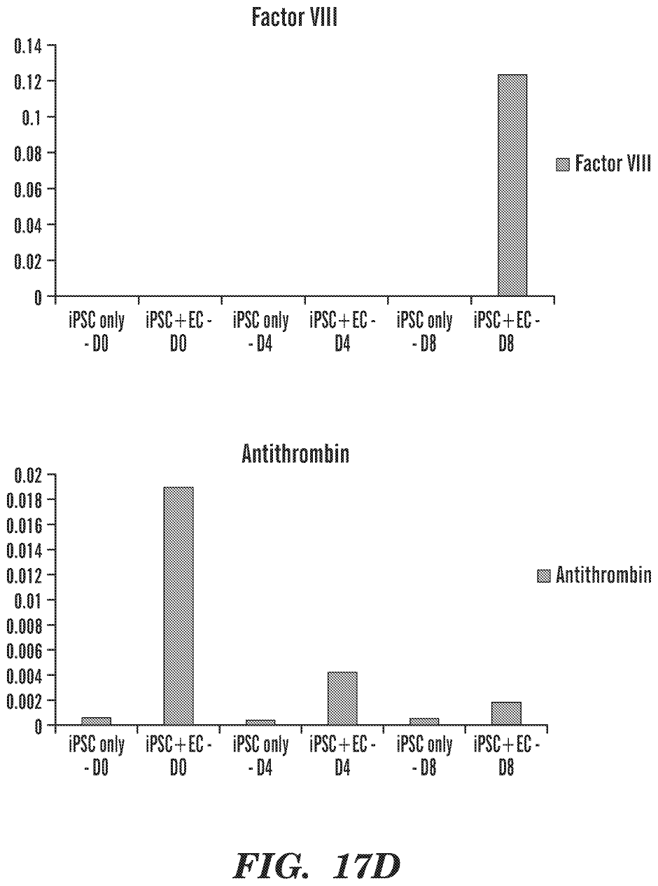

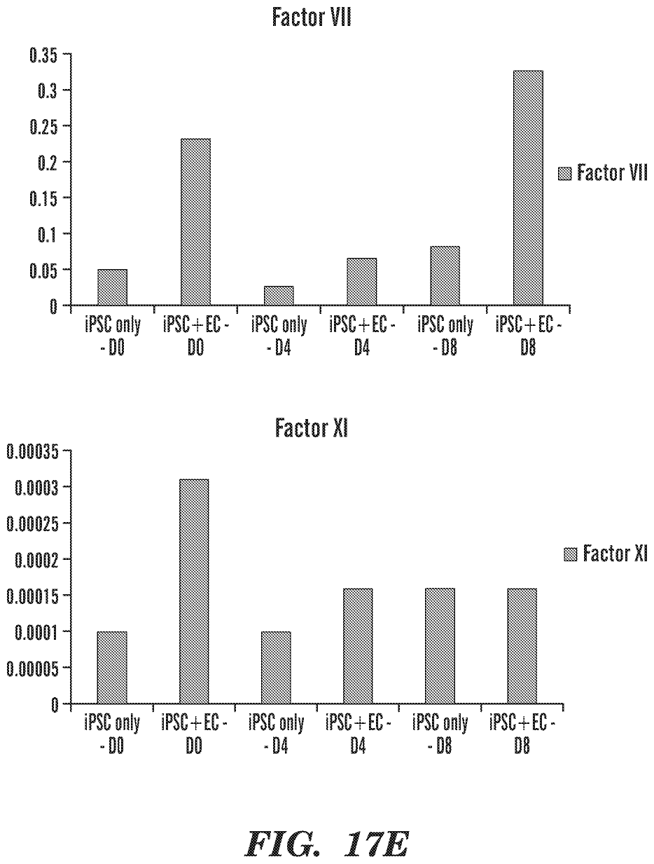

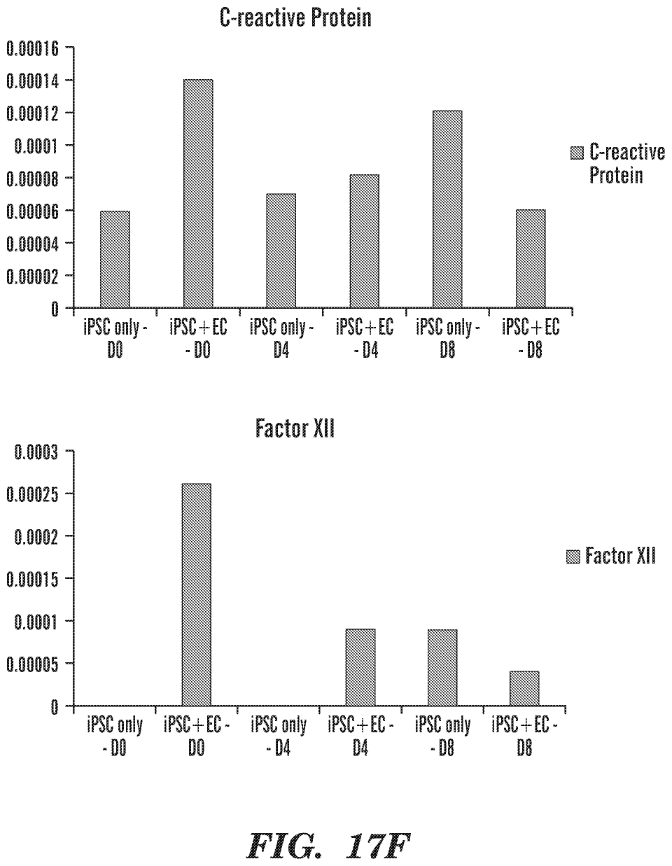

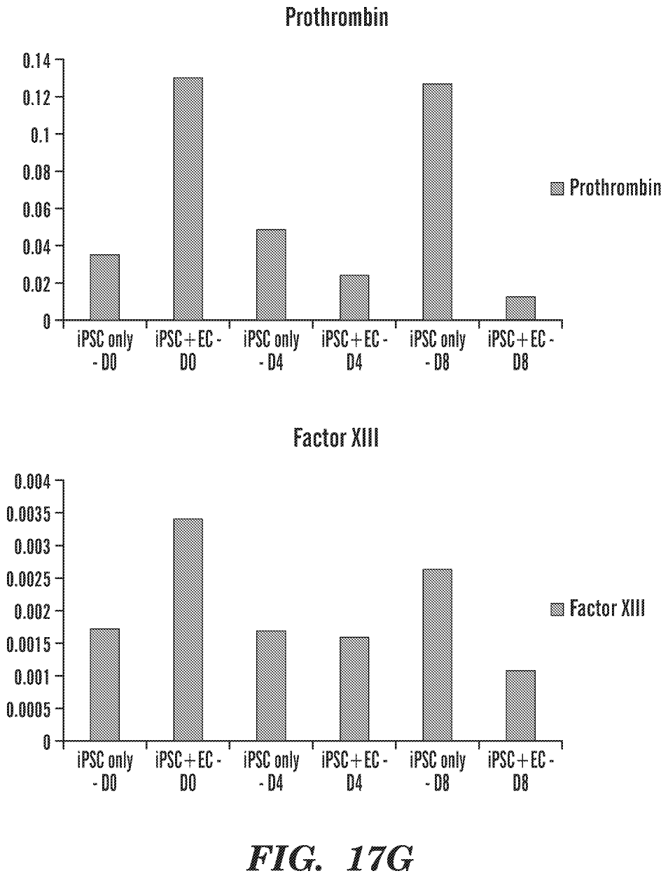

The studies of this example show that that HLCs with and without interlaced human endothelial cells are able to produce and secrete coagulation factors that are normally produced by both the primary hepatocyte and the endothelial cells in vivo. These coagulation factors include von Willebrand factor (vWF) and Factor IX (FIG. 17A), Protein C and Factor X (FIG. 17B), Protein S and Factor V (FIG. 17C), Factor VIII and Antithrombin (FIG. 17D), Factor VII and Factor XI (FIG. 17E), C-reactive Protein and Factor XII (FIG. 17F), and Prothrombin and Factor XIII (FIG. 17G). The HLCs with and without interlaced human endothelial cells are generated using the differentiation protocol disclosed herein. These HLCs allow for the treatment of patients suffering from blood coagulation disorders, such as hemophilia.

The results described above were carried out using the following methods and materials.

### Cell Sources and Culture Conditions.

Human induced pluripotent stem cells (hiPSCs) were a foreskin fibroblast-derived cell line iPS(foreskin)-3 (purchased from WiCell Research Institute, Madison, Wis.—cat # WB0002) and cultured in chemically defined stem cell medium (mTeSR1 basal medium with mTeSR1 supplement, Stem Cell Technologies, Ontario, Canada) on a Matrigel matrix (BD Biosciences, San Jose, Calif.). iPSC colonies were passaged using Versene (EDTA) (Lonza, Allendale, N.J.) for 8 minutes at room temperature.

### Embryoid Body (EB) Formation.

Agarose micro-well arrays were made using locally developed Teflon stamps and low melting point agarose (Sigma-Aldrich). The agarose, 40 g L⁻¹, was dissolved in phosphate buffered saline (PBS) at 100° C. and pipetted into the culture ware. The Teflon stamps were pressed into the agarose solution for approximately 5 minutes. The agarose gelled in about 2 minutes and the stamp was withdrawn with resultant microwell arrays in the agarose gel substrate. After the agarose gelled, arrays were primed by incubation with EB differentiation medium (1:1 mixture IMDM and F-12 Nutrient Mixture (Ham) (Invitrogen), 5% fetal bovine serum (Invitrogen), 1% (vol/vol) insulin transferrin selenium-A supplement (Invitrogen), 55 μM monothioglycerol (Sigma-Aldrich), 100 U L⁻¹ penicillin, and 0.1 mg L⁻¹ streptomycin (Invitrogen) overnight at 37° C. and 5% CO₂.

For hiPSC-EBs formation, 1.2×10 dissociated hiPSC in a 50 μl suspension were placed in each microwell array and allowed to sediment into the microwells. After 24-hour incubation at 37° C., three-dimensional EB were aspirated from the microwells and transferred to a 35 mm tissue culture dish (BD Biosciences). The cells were kept in suspension culture in basal hepatocyte medium under gentle agitation on an orbital shaker at 37° C. and 5% CO₂ with medium changes every other day.

### Hepatic Differentiation Procedure.

Our four-stage in vitro hepatic differentiation protocol sought to recapitulate the changes that occur during embryogenesis. The four stages are definitive endoderm, foregut endoderm, hepatobiliary progenitor and committed hepatocyte. Each stage of the differentiation protocol lasted four days with two every-other-day medium changes and addition of the soluble differentiation factors.

The basal differentiation medium consisted of IMDM with F-12 Nutrient Mixture (Ham), 5% fetal bovine serum, 1% (vol/vol) insulin transferrin selenium-A supplement, 55 μM monothioglycerol, 100 U ml⁻¹ penicillin, and 0.1 mg ml⁻¹ streptomycin (Sigma-Aldrich). Differentiation towards the definitive endodermal stage was promoted through the addition of 10 ng ml⁻¹ basic FGF, 100 ng ml⁻¹ Activin-A and 10 ng ml⁻¹ TGF-β (all from PeproTech, Rocky Hill, N.J.). The second foregut endoderm stage was promoted through the addition of 10 ng ml⁻¹ FGF-4 (PeproTech) and 10 ng ml⁻¹ BMP-4 (Invitrogen). Following endodermal commitment, absence of Wnt signaling is integral to hepatobiliary differentiation. The addition of Wnt pathway inhibitors, 1 μg ml⁻¹ WIF-1 (R&D System, Minneapolis, Minn.) and 0.1 μg ml⁻¹ DKK-1 (PeproTech), served to suppress Wnt signaling and promote the third stage of differentiation. Following hepatobiliary commitment, the presence of HGF and oncostatin determines differentiation into cholangiocytes or hepatocytes. By adding 50 ng ml⁻¹ HGF (PeproTech) and 30 ng ml⁻¹ Oncostatin A (PeproTech), we directed the hepatobiliary cells into a hepatocyte pathway at the fourth stage.

For the embryoid body differentiation, all factors were added to the cell culture media and the embryoid bodies were maintained in suspension through gentle orbital agitation. Before the application of the differentiation protocol, undifferentiated hEBs were collected from the same batch to be used as negative control. All the experiments for the hepatic differentiation with and without inhibitors were performed starting from the same batch of hiPSC-EBs; therefore, the samples were analyzed all at the same time at the end of the differentiation process to ensure the reproducibility of our results.

### hEB Viability.

At the end of the differentiation process, cell viability was evaluated by LIVE/DEAD staining (Catalog # L-7013, Molecular Probes) to determine the presence of any core necrosis according to the manufacturer's instruction. Fluorescent images were acquired with confocal microscopy using Olympus IX81.

### Gene Expression Assay.

Reverse transcriptase-PCR (RT-PCR) was performed to verify the presence of characteristic gene markers of differentiation. RNA was extracted using Trizol reagent (Invitrogen) and quantified by spectrophotometry (NanoDrop 2000, Thermo-Scientific). RNA was reverse transcribed to cDNA using the MMLV enzyme (Maloney Murine Leukemia Virus Reverse Transcriptase, Promega, Madison, Wis.). cDNA was amplified using Taq polymerase with the following parameters: one cycle of 94° C. for 4 min, 30-35 cycles of denaturation at 94° C. for 30 sec., and annealing at 60° C. for 30 sec. The following genes were evaluated: Alpha fetoprotein (AFP), Albumin, Cytokeratin 18, and P450 cytochromes Cyp3a4, Cyp2c9, Cyp3a7, Cyp1b1, Cyp2b6, Cyp1a2, CK-7, HNF-1 β, EpCAM, NCAM, Anion Exchanger 2 (AE2), SALL4 and Cyp3a7. GAPDH was used as the reference housekeeping gene. Values were normalized and reported relative to the glyceraldehyde-3-phosphate (GAPDH) housekeeping gene. Error bars represent the standard deviation of three independent experiments. Data is presented as mean±SD.

For quantitative RT-PCR (qRT-PCR), extracted RNA was treated with RNase-free DNase (Promega) and reverse-transcribed using an iScript cDNA synthesis kit (Bio-Rad) according to manufacturer instructions. Custom PrimePCR plates (Bio-Rad, 96 well, SYBR plate with 9 unique assays, Catalog #10025217) with lyophilized primers of interest were used with SsoAdvanced Universal SYBR green and run according to the manufacturer instructions. The following amplification conditions were used for a total of 40 cycles: activation for 2 minutes at 95° C., denaturation for 5 seconds at 95° C., annealing at 60° C. for 30-second melt curve at 65-95° C. (0.5° C. increments) for 5 sec/step. CFX96 Touch (Bio-Rad) was used for the amplification and data was processed using CFX Manager 3.1 (Bio-Rad). Values were normalized and reported relative to the glyceraldehyde-3-phosphate (GAPDH) housekeeping gene.

### Immunofluorescence Assay.

Embryoid bodies undergoing differentiation were collected at the end of each stage for immunofluorescence analysis of stage-specific markers. The embryoid bodies were fixed with 4% (wt/vol) paraformaldehyde for 90 minutes, permeabilized with 0.3% (vol/vol) Triton-X 100 in PBS for 1 hour, and blocked with 0.5% (vol/vol) goat serum (Sigma-Aldrich) in PBS for 1 hour. Samples were incubated with the primary antibody at 4° C. for three days. After several washes, the samples were then incubated with the secondary antibody at room temperature for 2 hours. The above incubation times were necessary for complete staining, likely due to the large radius of the EB clusters and increased time for diffusion.

The following human specific primary antibodies were used: rabbit anti SOX17 (Santa Cruz, sc-20099; 1:100); mouse anti FOXA2 (Abcam, ab60721); 5 μg ml⁻¹, goat anti Hhex (Santa Cruz, sc-15128; 1:100); mouse anti GATA-4 (Santa Cruz, sc-25310; 1:100); mouse anti AFP (Santa Cruz, sc-166325; 1:100); mouse anti HNF-4α (Santa Cruz, sc-8987; 1:100); goat anti Albumin (Santa Cruz, Santa Cruz, Calif., sc-46293; 1:100); mouse anti Cytokeratin 18 (CK-18) (Abcam, ab82254, 5 μg ml⁻¹); mouse anti HNF1-α (Santa Cruz, sc-135939; 1:100); and rabbit anti human C-MET (Santa Cruz, sc-10; 1:100). The following secondary antibodies were used: Cy2-AffiniPure goat to mouse IgG; Fc Subclass 1 Specific (Jackson ImmunoResearch, 1:100); Cy3-AffiniPure goat to rabbit IgG (H+L) (Jackson ImmunoResearch, 1:100) and Cy5-conjugated AffiniPure rabbit to goat IgG (Jackson ImmunoResearch, 1:100). Nuclei were counter-stained with 4′6-diamidino-2-phenylindole (DAPI) in PBS for 1 hour. Fluorescent images were acquired with confocal microscopy using Olympus IX81. The yield of albumin-producing cells obtained with our differentiation protocol was determined by counting the number of albumin-positive cells over the total number of cells in each optical cross-section using a confocal microscope, and averaged over a minimum of 10 microscopic fields for each cluster and a minimum of 50 different clusters per differentiation condition.

### FACS Analysis and Cell Sorting.

After completion of the differentiation protocol, 100 hiPSC-EB-HLC with and without inhibitors were digested using trypsin for 15 minutes at 37° C. Live/Dead Yellow Fixable Stain was utilized to assess viability. Intracellular Albumin staining was performed using the Fixation/Permeabilization Staining Buffer Set (eBioscience, San Diego, Calif.). For the FACS analysis the following monoclonal antibody was used: anti-Human Serum Albumin APC-conjugated Antibody (R & D SYSTEMS). Data acquisition was performed on BD FACS Aria II instrument. Purity after sorting was routinely>95%. We drew the threshold based on the control sample stained for viability (live/dead stain) but not for albumin. A gate was drawn so that the frequency of this control sample was considered the zero. All the events above the threshold in the stained samples were deemed as positive. Analysis was performed using FlowJo software. Mean fluorescence intensities (MFIs) were calculated using the geometric mean of the appropriate fluorescence channel in FlowJo. Expansion Indices were determined using the embedded FlowJo algorithm.

### Albumin, AFP and Fibrinogen Secretion Assays.

After 24 hours of the last change of medium, conditioned medium coming from fully differentiated hEBs was collected and stored at −80° C. Albumin secreted from the differentiated embryoid bodies into the culture media was quantified using a Human Albumin ELISA kit (Abcam ab108788) according to the manufacturer's instructions. For the Alpha-fetoprotein secretion assay the quantification was performed using an Alpha Fetoprotein Human SimpleStep ELISA kit (Abcam ab193765) according the manufacturer's instructions. The Fibrinogen secretion into the culture supernatant was quantified using a Fibrinogen Human SimpleStep ELISA Kit (abcam-ab171578) following the manufacturer's instructions. All the samples were carried out in triplicate.

### Intracellular Urea Content Assay.

Total Urea content within the differentiated hEBs was performed using the whole clusters that were digested with a specific buffer coming from a commercial Urea Assay Kit (abcam-ab83362) according to the manufacturer's instructions.

### Indocyanin Green Uptake and Release Assay.

Fully differentiated hEB were incubated with indocyanin green (IGC, Sigma-Aldrich) in basal medium for 1 hour at 37° C. according to the manufacturer's instructions. Uptake of IGC was detected with light microscopy using an Olympus IX81. IGC release was detected 6 hours later to ensure that all the positive cells released the IGC.

### Uptake of Low-Density Lipoproteins (LDL) Assay.

LDL uptake assay was performed after completion of the differentiation protocol using Dil-Ac-LDL following the manufacturer instruction. (Alfa Aesar-J65597). Briefly, the cells were incubated overnight in serum free pre-incubation media containing 0.1% BSA. The next day the differentiated hEBs were incubated for 5 hours at 37° C. with Dil-Ac-LDL 10 μg/mL in pre-incubation media. After the incubation the cells were washed several times with pre-incubation media and fixed with 4% paraformaldehyde for 1 hour. DAPI staining for the nuclei was performed after fixation for 1 hour at RT. Fluorescent images were acquired with confocal microscopy using an Olympus IX81.

### Periodic Acid-Schiff (PAS) Staining.

The glycogen storage of differentiated hEBs was evaluated using PAS staining according to the manufacturer instructions (Sigma-Aldrich). Briefly, the clusters were fixed with 4% paraformaldehyde for 1 hour, then oxidized for 5 minutes with Periodic Acid solution and then washed several times. Following the washes, 15 minutes incubation with Shiff Reagent was performed followed by color development with dH2O for 5 minutes. Staining was detected with light microscopy using an Olympus IX81.

### Oil Red Staining.

After differentiation, the cells were tested for the lipid vesicle storage using Oil Red O staining according to the manufacturer's protocol (abcam-ab 150678). Briefly, the clusters were fixed with 4% paraformaldehyde for 1 hour, and then incubated for 2 minutes with Propylene Glycol followed by a 6 minute incubation with Oil Red O solution. After the staining, 1 minute incubation with 85% Propylene Glycol was performed followed by 2 washes with dH2O. Staining was detected with light microscopy using an Olympus IX81.

### CYP Activity Assay.

The Cytochrome P450 enzymes activity was performed using the P450-Glo™ Assay Kit (Promega, Madison, Wis.) according to the manufacturer's instructions. We tested the activity of different P450 enzymes, in particular the CYP2B6 (P450-Glo CYP2B6—V8321/2—Promega, Madison, Wis.), CYP3A4 (P450-Glo CYP3A4 (Luciferin-IPA)—V9001/2—Promega, Madison, Wis.), and the CYP1A2 (P450-Glo CYP1A2 Induction/Inhibition—V8421/2—Promega, Madison, Wis.) by incubating them with different inducers. For the CYP2B6 activity assay, undifferentiated hiPSC, primary hepatocytes and differentiated HLCs were incubated with basal medium containing 1000 μM Phenobarbital solution (Sigma), or DMSO (0.1%) for 48 hours. For the CYP3A4 activity assay, undifferentiated hiPSC, primary hepatocytes and differentiated HLCs were incubated with basal medium containing 20 μM Rifampicin solution (Sigma), or DMSO (0.1%) for 48 hours. For the CYP1A2 activity assay, undifferentiated hiPSC, primary hepatocytes and differentiated HLCs were incubated with basal medium containing 50 μM Omeprazole solution (Sigma), or DMSO (0.1%) for 48 hours. Measurement of the activity of each enzyme was performed by reading the luminescence using a luminometer (Synergy H1 Hybrid Reader—Biotek) according to the manufacturer's instructions. All the experiments were performed in triplicate.

### Ammonia Metabolism Assay.

Ammonia metabolism was evaluated by changes in ammonia concentration in the cell culture supernatant over a 24-hour period after addition of ammonium chloride. 1 mM of ammonium chloride standard was added to the culture dishes containing 100 differentiated embryoid bodies in suspension. Supernatant was collected and ammonium concentration was measured at 1-, 6- and 24-hour intervals after ammonium chloride addition using a colorimetric ammonia assay kit (BioVision, Milpitas, Calif.).

### Cell Transplantation.

The Institutional Animal Care and Use Committee (IACUC) approved the use of animals for experimentation in this study. Acute liver failure was induced in 270-350 g athymic nude rats (Crl:NIH-FoxnI^{rnu}, Charles River Laboratories, Wilmington, Mass.) by intraperitoneal injection of 950 mg kg⁻¹ of sterile D-galactosamine dissolved in Hanks Balanced Salt Solution (Sigma-Aldrich). First, 3D clusters (organoids) are collected from the culture conditions described above and washed with D5LR solution. The clusters may contain hiPSC only, or a mix of hiPSC+EC+MSC. No Rock inhibitors were used to prepare the 3D clusters. Under inhalational anesthesia, 80-100 hiPSC-EB-HLCs were injected into the spleen body as 3D clusters through the caudal pole of the spleen. Following injection, the caudal pole was ligated. The experimental groups consisted of animals transplanted with the hiPSC-EB-HLCs treated with inhibitors and hiPSC-EB-HLCs treated without inhibitors. Negative controls consisted of animals that received hepatocyte medium only and animals transplanted with undifferentiated hiPSCs embryoid bodies. Healthy controls consisted of animals without liver injury transplanted with hiPSC-EBs. The animals were monitored daily and received standard chow and water ad libitum. Animal survival was tracked as a primary end point. Animals were sacrificed after 14 days or earlier if they had moribund appearance or greater than 30% body weight loss in accordance with predefined humane care criteria. All experiments were carried out in accordance with the approved IACUC guidelines.

### Serum Analysis.

The tail vein was phlebotomized prior to transplantation, 48-72 hours after transplantation, and at time of sacrifice. Concentration of serum alanine transaminase (ALT) in whole blood was measured using VetScan 2.0 (Abaxis, Union City, Calif.). The presence of human albumin in the rat serum was evaluated using a Human Albumin ELISA Quantitation Set that was non-cross reactive with rat albumin (Bethyl Laboratories).

### Histology and Immunohistochemistry.

Liver and spleen samples were recovered at sacrifice or death and fixed with 10% neutral buffered formalin. The cells were subsequently embedded in paraffin and sectioned with hematoxylin and eosin staining for histologic assessment.

The paraffin-embedded slides were deparaffinized using xylene-substitute and ethanol and immunohistochemistry was performed on rat liver and spleen sections to identify the presence of human albumin. Following deparaffinization, endogenous peroxidase activity was blocked with 4% hydrogen peroxide.

For human albumin detection, non-specific binding was blocked with 2% donkey serum for 60 minutes (Sigma-Aldrich) and the slides were incubated with a non-cross reactive goat antibody to human albumin primary antibody (Bethyl Laboratories; 1:500) for 60 minutes. The secondary antibody used was HRP-conjugated donkey antibody to goat IgG (Santa Cruz; 1:200) for 60 minutes.

For the immunofluorescence staining of the rat liver sections, the slides were fixed with 4% (wt/vol) paraformaldehyde for 30 minutes, permeabilized with 0.3% (vol/vol) Triton-X 100 in PBS for 30 minutes, and blocked with 0.5% (vol/vol) goat serum (Sigma-Aldrich) in PBS for 1 hr RT. Samples were incubated with the primary antibody at 4° C. overnight. After several washes, the samples were then incubated with the secondary antibody at room temperature for 1 hour. The following human specific primary antibodies were used: mouse anti human HNF-3β (RY-7) (Santa Cruz, sc-101060; 1:100) and rabbit anti human C-MET (Santa Cruz, sc-10; 1:100).

### Statistical Analysis.

Quantitative data are expressed as mean±standard deviation. Comparisons were made using Fisher's exact test or Chi-square tests for categorical variables, and Student t tests or analysis of variance for continuous variables. All statistical analyses were performed using JMP 9.0 (Stata Corp LP, College Station, Tex.).

### Protocol for Preparing Hepatocyte-Like Cells for Transplantation

Immediately following differentiation, Hepatocyte like cells are re-suspended in the Transplant Media, which is Lactated Ringer's Solution with 5% Dextrose (D5LR+5% dextrose). Following resuspension, the trypan blue exclusion assay is used to determine the cell viability. The cell viable to nonviable ratio provided exact cell numbers. Human Hepatocyte-like Cells intended for transplantation (after perfusion and isolation), were prepared into a final cell mixture, and the cells were then resuspended into the Transplant Media.

Once the Attending Physician and Attending Radiologist have determined the site at which the prepared human hepatocytes would be delivered, the site is prepared for the injection of the prepared hepatocytes. Based on the patients disease and a decision of the best site for cell infusion, the Attending Physician will instruct the Interventional Radiologist as to where the catheter will be inserted and placed for either splenic or intra portal administration of hepatocytes and hepatocyte like cells (HLCs). The sterile cells are then gently moved into a sterile glass tube and injected slowly with a rocking gentle motion to evenly distribute the cells in the buffer comprising D5LR+5%. The flow of the cellular mixture into the portal vein or splenic artery is monitored. The Attending Physician and Attending Radiologist will determine the safest and best route for cell administration as well as catheter introduction as previously documented with human hepatocyte transplant practice. (Strom S. C. et al., Transplantation. 63(4):559-569, 1997).

Cryopreservation of Human Hepatocytes. Hepatocyte-like cells not used immediately for research or clinical cell transplantation may be cryopreserved. To cryopreserve cells, the cells were frozen in University of Wisconsin (Belzer's) Solution (VIASPAN) and were supplemented with 10% DMSO (Sigma). The hepatocyte/cell freeze solution mixture was then placed into freezing vials (2 ml, 5 ml or 13 ml) or freezing bag, and these vials/bags of this hepatocyte/cell freeze solution mixture was placed directly into a −20 freezer for a period of 2-3 hours. The vials/bags were then transferred to a −70 to −80 degrees Celsius freezer. Upon approval for cell release for possible patient cell transplant in the clinical setting, the frozen hepatocytes could be placed into the Cryoplus liquid nitrogen tank (LN2) for long term storage if the cells were not used within 6 months.

23. Hepatocyte Lot Release Criteria is as follows:
a. Viability Test=Trypan Blue Assay=≥70%. Before Lot Release
b. Yield Test=Trypan Blue Assay=≥250×10\hat{ }6 total cell volume. Before Lot Release.
c. Gram Stain Test=Gram's method (crystal violet, Gram's iodine)=negative. Before Lot release.
d. Sterility=Per USP 71 and 21 CFR 610.12. Negative. Negative at 24 hours report. Results also at day 7 and day 14 will follow.
e. Cell Identity=Microscopic examination (10× and 40× computer imaging). Cell identity. Before lot release.
f. Cell function=CYP P450 luminescent assay. Function determination. Following.
g. Endotoxin=Quantitative Limulus Amebocyte Lysate Test. ≤EU of final product volume/kg recipient body weight, done before lot release in house. (as of 2011 this no longer is required as an FDA recommended lot release test, however, still recommended as a test.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for generating hepatocyte-like cells the method comprising
(a) incubating induced pluripotent stem cell in a round bottomed convex well comprising agarose to generate a spherical embryoid body;
(b) contacting the embryoid body with one or more differentiation factors selected from the group consisting of basic FGF, Activin-A and TGF-β, thereby forming an embryoid body comprising definitive endoderm cells;
(c) contacting the embryoid body of step b with FGF4 and/or BMP-4 to form an embryoid body comprising foregut endoderm cells;
(d) contacting the embryoid body of step c with a Wnt pathway inhibitor to form an embryoid body comprising hepatoblast cells;
(e) contacting the embryoid body of step d with a HGF and/or Oncostatin A to form an embryoid body comprising mature hepatocyte-like cells.

2. A method for generating hepatocyte-like cells the method comprising
(a) incubating an induced pluripotent stem cell and an adipose-tissue derived endothelial cell in a round bottomed convex well comprising agarose to generate a spherical embryoid body;
(b) contacting the embryoid body with one or more differentiation factors selected from the group consisting of basic FGF, Activin-A and TGF-β, thereby forming an embryoid body comprising definitive endoderm cells;
(c) contacting the embryoid body of step b with FGF4 and/or BMP-4 to form an embryoid body comprising foregut endoderm cells;
(d) contacting the embryoid body of step c with a Wnt pathway inhibitor to form an embryoid body comprising hepatoblast cells;
(e) contacting the embryoid body of step d with a HGF and/or Oncostatin A to form an embryoid body comprising mature hepatocyte-like cells.

3. The method of claim 1, wherein the definitive endoderm cells express SOX17 and FOXA2; wherein the foregut endoderm cells express HHEX and GATA4: wherein the hepatoblast cells express AFP and HINF-4α.

4-5. (canceled)

6. The method of claim 1, wherein the hepatocyte-like cells express one or more of the following markers albumin, HNF-1α, C-MET, and CK-18.

7. The method of claim 1, wherein step b comprises contacting the embryoid body with basic FGF, Activin-A and TGF-β or FGF4 and BMP-4.

8. (canceled)

9. The method of claim 1, wherein step c comprises contacting the embryoid body of step c with WIF-1 and DKK-1.

10. The method of claim 1, wherein the hepatocyte-like cells express five P450 isoforms Cyp1B1, Cyp2C9, Cyp3A4, Cyp2B6 and Cyp3A7 or express Alpha fetoprotein, Albumin, and CK18.

11. (canceled)

12. The method of claim 1, wherein the hepatocyte-like cells of step d form a cluster that is 800-1,000 μm, but that shows no core necrosis.

13. (canceled)

14. The method of claim 1, wherein the hepatocyte-like cells are capable of ammonium metabolism, of detoxification as measured by increase in CYP isoform gene expression, secretion of Albumin, Alpha Fetoprotein and/or fibrinogen, glutathione, a coagulation factor, and/or comprise intracellular Urea.

15-17. (canceled)

18. The method of claim 1, wherein the method generates 80% or more hepatocyte-like cells.

19. The method of claim 1, wherein the induced pluripotent stem cell and adipose-tissue derived endothelial cell are mammalian cells.

20. The method of claim 1, wherein the induced pluripotent stem cell and adipose-tissue derived endothelial cell are rodent or human cells.

21. The method of claim 1, wherein the induced pluripotent stem cell is derived from an amniotic cell.

22. (canceled)

23. The method of claim 1, wherein the method further comprises coating the cluster with a hydrogel and culturing the coated cluster with mesenchymal stem cells, thereby forming a mesenchymal layer of cells around the cluster.

24. (canceled)

25. The method of claim 1, wherein the hepatocyte-like cell is capable of functioning in the Liver phase 1 and/or Liver phase 2 detoxification pathway.

26-28. (canceled)

29. A method for treating a blood coagulation disorder, the method comprising administering to a subject having the blood coagulation disorder a hepatocyte-like cell produced according to the method of claim 1.

30. The method of claim 29, wherein the hepatocyte-like cell secretes a coagulation factor.

31-32. (canceled)

33. A method for treating liver disease or dysfunction, the method comprising administering to a subject having liver disease or dysfunction a hepatocyte-like cell produced according to the method of claim 1.

34-35. (canceled)

36. A cellular composition comprising a hepatocyte-like cell produced according to the method of claim 1 or 2 and an excipient.

37-41. (canceled)

42. A kit comprising a hepatocyte-like cell produced according to the method of claim 1 and instructions for the administration of said cell.
